SEQUENCE LISTING (105 sequences)

SEQ ID NO: 1 - protein, 1278 aa, unidentified
MYNLELFTGKYQRQLTLRNALIPVGATLENLKKHGIITTDEELEKKYQEAKAIIDDYHRHFIDANLSRIALDWQELAEALLAYQHNRNNETTEKLEKVQATYRKQLLKAFSEDENYKHLFKKELFTKVLPSYYTDKEQLAVINAFKNFFTYFSGLNENRANIYSTDPKSTAIGYRLIHENFPKFITDCAIFAVLQQHCPQVITDTETVLRQLAIIEADTELADYFLPNSFNAFLTQEGISKFNNIIGELNKNINLAMQQDASLKPSLRSKRATKMFILYNQILSKDDSLPFIDVFENDNQLLTALQQFYTCISAPDGTLNKAAFLFKTLETCQKDKIYISGKDINSLSKALYGGSNWNTLREAILTALQNEKEYKKLLKENDEAAIDRQLSKNYFSLDFLQTHADNLLITKIAEYFQTQHELAQQAVQTDLLGTQNLKATVKKSLDALLDYYRFISLFKTDDSLDKDPEFYGSYEEILSQLKEIVYIYNKTRNYMTKKPFNEEKFKLNFGNAQLAGGWDANKEADYTSIILTKDSAYYLGIMNKNSKPDIDTAITESTDNTYRKMVYKYFPDFSKMFVKCTMVKEVKNHFQENSTDFILQTNSFIKPLKITKEIYDLYSTEYDGKKKFQIDYLRRTGDAQGYYDALNKWIGFALAFMRSYDSTSIYDISQIEATQYERLDTFYKDINNICYNISFVNLAADKVDKWVAEGKLYLFKIYNKDFAPGRSGTKNLHTLYLESIFDEQNLKDVVVKLNGGAELFYRRKTKGNPSVHSKGSKLVNRTTADGQTIPEEIYQEIYLYANGNKEALSQQAQEYYPRAVIKTATHDIVKDRRYYEDKFFFHFPITINYKAPDSSGAFNQEVLAYLHHNPDVNIIGIDRGERNLIYMTVINQQGQILASKSFNSISQQAGTVNKSVDYHDKLDKRELERTEARKSWETIGKIATLKEGYLSAVIHEIAKYMVKYNAIVVMEDLNAGFKRMRGGIAEKAVYQKFEKMLIDKLNYLVFKNLPANEPGGVLKGYQLSSKFDSFEKMGKQNGFLFYVPAAFTSKIDPVTGFSSVFKFKEYTANTEKAKSFFTSFDSIVYSSEKDVFTFSFDYDNFKTYANMYRKKWQVSSYGERIKHEKNVIGHYIDSFVNPTQDIKTLFDTQNIDYSSEQNILPQLLQIEDNKASHSFWKGMLSAFQRIVQLRNSSRTLTTDGKETDYIISPVADSSGSYFDSRTADSALPQDADANGAYHIALKGLIYLNINNAAKDNKPKLYVKNEDWFKFTQERNSAK

SEQ ID NO: 2 - DNA, 3837 bp, unidentified
  source 1..3837: mol_type = genomic DNA
atgtacaacttagagcttttcaccggcaaataccaacgtcaacttacgcttcgcaatgcactcatccccgttggcgcaacgctggaaaacctcaaaaaacacggtattataacaactgatgaagaattagaaaagaaatatcaggaagctaaagctatcattgacgattatcaccgtcattttattgacgccaatctttcacgtattgcgttagactggcaggagctggcagaagcactgcttgcttaccagcataatcgtaataacgaaacaacagaaaaactggaaaaagtccaggctacatatagaaaacagctgcttaaagctttcagtgaagacgaaaactataagcacctgttcaagaaagagctttttaccaaagttctgccctcttactataccgataaagaacagcttgcagttatcaatgcttttaaaaactttttcacctatttcagcggcttaaatgaaaacagagccaatatttacagtacggatcctaaaagcacagctatcggttatcgacttatacatgaaaactttcctaaattcattaccgactgcgcaattttcgcagtgttgcagcaacactgcccacaagtcatcactgatacagaaactgtactgcgacagcttgcaataatagaagctgatacagaactggcagattacttcctgcccaacagctttaacgcttttctcactcaggaaggcatcagcaaatttaacaatatcataggagagctgaataaaaatatcaatcttgccatgcagcaggatgcaagtctgaaaccttctctgcgcagtaagcgtgcaacaaaaatgtttattctctataatcaaatcctgtctaaagatgactcacttccttttattgatgtctttgaaaacgataatcagctgcttaccgccctgcagcaattttatacctgtatttctgcaccggatggaacactgaataaagcagcatttttatttaaaacattagaaacctgtcaaaaagataaaatctacattagcggtaaagatatcaacagcctttctaaagctctttacggcggcagcaactggaatactttacgtgaagctattctaacagcactgcaaaatgaaaaggaatacaaaaaactgctgaaagaaaatgacgaagctgccattgaccgacagctcagcaagaactatttctctttagatttcctgcaaactcatgctgacaatctgcttataactaaaatcgccgaatacttccaaacccagcatgaactggcacaacaggcagtgcaaacagacctccttggtacgcaaaatcttaaagccacagtaaaaaaatcattggacgctttacttgattactaccgtttcatttctctgtttaaaactgatgacagtcttgataaagatccggaattttacggcagctacgaagaaatcctcagccaactgaaagaaatagtttacatctataacaaaacccgcaactacatgacgaaaaagcctttcaacgaggaaaaattcaagcttaattttggtaatgctcagcttgcaggcggttgggatgccaacaaagaagctgactacacttctatcatccttacgaaagacagtgcctattatctgggcattatgaacaaaaacagcaaaccagatattgatactgccattaccgaaagtactgacaatacctaccgcaaaatggtgtataaatacttcccagatttcagtaaaatgtttgtcaaatgtactatggtaaaagaagtaaaaaatcattttcaggaaaattccacagactttatcctgcaaacaaattcttttatcaagccattaaaaattaccaaagaaatatatgatctatacagtacagaatacgatggcaagaaaaaatttcagatagattatctgcgtaggactggcgatgctcaaggctattatgatgccttaaataaatggattggctttgctcttgctttcatgcgttcttatgacagcacttctatttacgatatttcacaaatagaagcaacgcaatatgaaaggctggacaccttctataaagacatcaacaacatctgctacaatatatctttcgtaaatcttgccgcagataaagtcgataaatgggtagcagaaggcaaactgtacctgtttaaaatttacaacaaggattttgctcccggacgtagcggtacaaaaaatctgcacaccctgtatctggaaagcatttttgacgaacagaatctgaaagacgttgttgtcaaacttaacggcggcgccgaacttttctaccgccgcaagactaaaggcaacccgtccgttcacagtaaaggcagtaaacttgtcaatcgtaccacagctgacggacagactattccggaagaaatctatcaggaaatctacctgtatgctaatggcaataaggaagcactcagccagcaggcacaggaatactatccccgcgctgttatcaaaaccgcaactcatgatattgtcaaggacagacgttattatgaagataaattcttcttccacttccccatcactataaattataaagcgccagacagttccggcgcctttaatcaggaagtgcttgcttacctgcatcacaatccagacgttaatatcatcggcattgaccgcggtgaaagaaaccttatctatatgactgtcatcaaccagcagggacaaatcttagccagcaaatcctttaacagcatcagtcagcaggcaggtactgtaaataaatctgtcgattaccacgataagctggataaacgagaacttgaaagaacggaagcccgaaaaagctgggaaaccatcggcaaaattgctactttaaaagaaggctatctttctgccgtcatccacgaaattgccaaatatatggttaaatataatgccatcgtcgttatggaagacctgaacgcaggctttaaacggatgcgcggtggcattgctgaaaaagctgtttaccagaaatttgaaaaaatgctgattgacaaactcaactacctcgtctttaaaaatttacctgccaacgaacccggcggcgtactgaaaggctatcagctgagcagcaaatttgacagctttgaaaaaatgggtaagcaaaatggtttcctgttctacgtgcctgccgcttttacctccaaaattgaccctgttaccggattcagcagcgtctttaaatttaaggaatacactgccaatacagaaaaagccaaaagcttctttaccagctttgacagtatcgtctatagctccgaaaaagacgttttcactttcagcttcgattacgataatttcaaaacctatgccaatatgtaccgtaaaaaatggcaggtaagctcctatggtgaacgtatcaagcatgaaaaaaatgttattggacattatattgacagctttgtaaatcctacgcaggatatcaaaacgctgtttgacacacaaaatatagattacagcagcgagcaaaatattctgccgcagctgctgcaaattgaagataataaagcaagccacagcttctggaaaggtatgttatcagctttccagcgtatcgtccagttaagaaacagcagcaggacgcttaccacagacggtaaagaaaccgattacatcatctctcccgttgccgacagttccggcagctattttgattcccgcactgcagattcagctctgccgcaggatgcagatgccaacggagcctaccatattgcgctaaaaggtctgatttatcttaatatcaataacgctgccaaagataataagccaaaactttatgttaaaaatgaagattggtttaaatttactcaggaaagaaattctgccaaataa

SEQ ID NO: 3 - DNA, 3837 bp, synthetic construct
  source 1..3837: mol_type = other DNA
atgggctataatctagaattatttacaggaaaatatcaacgtcagttaaccctgcgtaacgcgcttatcccggtgggtgccacgttggagaacctcaagaagcacggcattatcaccacagacgaagagttggagaagaagtaccaagaagcgaaagctatcatcgacgactaccatcgtcatttcatcgacgcgaacctgagccgcattgcgctggactggcaggagctggcggaagcactgctggcctaccaacacaaccgtaacaacgaaaccaccgagaaactggaaaaagtacaagcgacttaccgcaaacagctgctgaaggcgttttcagaagatgaaaactacaaacacctgttcaagaaggagctgtttacgaaggtgctgccaagttattacaccgacaaggagcagctcgctgtcatcaatgcctttaagaacttttttacctacttcagcggtctgaatgagaaccgcgcaaatatttacagcaccgaccctaaaagcaccgccatcggctatcgtctgattcatgagaattttccgaaattcatcaccgattgtgcgattttcgcggttctgcaacagcactgtccgcaggttatcaccgacacagagaccgtgttgcggcaattggcgattattgaggcggacactgagctggccgactactttctgccaaattcgttcaacgcgttcctgacccaggagggcatcagcaagtttaataacatcattggtgagctgaacaagaacatcaacctggcaatgcaacaagacgcctcactaaagccgtctctgcgtagcaaacgcgcgactaagatgtttatcctctacaaccagattctgagcaaggacgacagcttaccgtttattgacgtttttgagaacgacaaccagttgttgaccgcactccaacaattttatacctgcattagcgcaccggatggtactttaaacaaggccgcgttcctgttcaagaccttggaaacgtgccagaaagataaaatttacattagcggtaaggacattaattcgctgagtaaagcattgtacggcggttccaattggaatactctgagagaagcgattctgactgcgctgcagaacgaaaaagaatacaaaaaattgcttaaagagaacgatgaagctgctatcgaccgccagctgtctaagaactatttcagcctcgattttctgcagacccatgcggacaatctgctgattaccaaaatcgccgagtacttccagacgcaacacgagctggcgcaacaggctgtgcagaccgacctactgggtacacaaaacctcaaggccacggtcaaaaagtccctggacgcgctgttagattactaccgttttatcagcttgttcaagaccgatgattcgctggacaaggatccggaattttacggctcttacgaagaaatcctgagccaattgaaagaaattgtctatatttacaacaaaacccgtaactacatgactaagaaaccgtttaacgaggagaagttcaagctgaacttcggcaatgcgcagctggcgggtggctgggatgcgaataaagaagctgattacacctccatcatcctgaccaaggactccgcgtactatctgggcataatgaataaaaatagcaaaccggatatcgataccgcaatcaccgagagcaccgataatacgtaccgcaaaatggtctataaatacttcccggatttttctaaaatgtttgtgaagtgcacgatggtgaaagaagttaaaaatcattttcaagagaatagcactgactttattttgcaaaccaacagcttcatcaagcctctgaaaataaccaaagagatttatgacctgtatagtactgaatacgatggcaagaagaaattccagatcgactacctgcgtcgtacgggtgatgctcaaggttattacgacgcgttgaacaaatggatcggcttcgcgctggcgtttatgcgtagctacgattccacgagcatttacgacatttcccaaattgaggcgacgcaatatgaacgtctggataccttttataaggacattaataacatctgctataacatctccttcgtgaatctagcagcagataaagttgataaatgggtagccgaaggtaagttatatctgttcaaaatctataataaagacttcgccccaggtcgtagcggcaccaagaacctgcacaccttgtacctggagtcgatctttgacgagcaaaacctgaaagatgttgtggtgaagttgaacggtggtgcggagctgttctaccgtcgtaaaaccaaaggtaatccgagcgttcacagcaaaggttctaagttggttaaccgcaccaccgctgacggccagaccatcccggaagagatctatcaagagatctacttgtacgcaaacggcaacaaagaggcgctcagccagcaagcgcaggagtattacccgcgtgctgtgattaaaaccgcgacgcacgatatcgttaaggacagacgttattacgaagacaagttctttttccatttcccgattaccattaactataaagctcccgattcctcaggcgcgttcaaccaggaggttctggcttatcttcaccacaacccggacgtaaacatcattggcattgacagaggggaacgaaacctgatttacatgaccgttatcaatcagcagggtcagatcctggcgtctaagtccttcaattctatcagccagcaagccggtaccgtgaataaaagcgtggattaccatgataagttggacaaacgtgagctggaacgtacggaggcacgtaaaagctgggaaacgatcggtaagatcgcgaccctaaaagaaggttacctgagcgcggtcatccacgaaattgcaaagtatatggtcaaatataatgcaattgtggttatggaagatctcaacgctggttttaagcgcatgcgtggtgggattgcggaaaaagcggtctaccagaaattcgagaaaatgcttatcgataagctgaactacctggtttttaagaatctgccggcaaacgagccgggcggtgttctgaaaggctatcagctgtctagtaagttcgatagcttcgagaagatgggtaaacagaacggttttttattttatgtgccggcagcgttcaccagcaaaattgacccggtgacgggcttctcttccgtgttcaaattcaaagaatatactgcgaacaccgagaaggccaagtccttcttcacctcgttcgactctatcgtgtattcgtccgagaaagacgtattcacgtttagctttgactatgataattttaaaacctacgccaacatgtatcgcaaaaaatggcaggttagctcatacggcgaacgcattaagcacgaaaagaacgtgattggccattatattgattcgttcgtgaacccgacccaagatattaaaaccctgttcgatacccaaaatatcgactatagctcggaacagaatatcctaccgcagctgctgcaaattgaggacaataaagcaagccatagcttctggaaaggcatgctgagcgccttccaacgcatcgttcagctacgtaattcctcccgtacgttgaccacggacggtaaggagacggattatattatttcgccggttgcggatagctctggtagctactttgacagccgcacagctgacagcgcactgccgcaggatgcggacgctaacggcgcgtatcacattgccctgaagggcctgatctatttgaacatcaacaacgcggctaaggacaacaagccgaaattgtacgttaagaacgaggattggtttaaattcacccaggagcgtaatagcgctaag

SEQ ID NO: 4 - DNA, 3831 bp, synthetic construct
  source 1..3831: mol_type = other DNA
tacaatctggagctgttcaccggcaagtaccagagacagctgacattgagaaacgccctcatccctgtgggcgccaccctggaaaacctgaaaaagcacggcatcatcaccactgacgaagaacttgagaaaaaataccaggaggcaaaggcgatcatcgatgactaccatagacacttcatcgatgccaacctgtccagaatcgccctggactggcaggagctggccgaggcccttctcgcctaccagcacaaccgcaacaacgaaacaacagaaaaactggagaaggtccaggccacataccggaaacaactactgaaggccttcagcgaggacgagaactacaagcacctgttcaagaaggaactgttcaccaaggtgctgcctagctactacacagataaggaacagttagccgtgatcaacgcctttaaaaacttcttcacctactttagcggcctgaacgagaacagagccaacatctacagcaccgaccccaagagcaccgccatcggctacagactgattcacgaaaacttccctaagttcatcactgactgcgccatattcgccgtgctgcaacagcattgtcctcaggtgatcaccgatacagagacagtactgagacagctggccatcatcgaggccgataccgagcttgccgattacttcctgcccaacagcttcaacgcttttctgacccaggagggcatcagcaagttcaacaacatcatcggcgaactgaacaagaacatcaatctggccatgcagcaggacgcatctctgaaaccatctctgagaagcaagagagctacaaagatgtttatcctgtacaaccagatcctgagcaaggacgacagcctgcctttcatagacgtgttcgagaacgacaatcagctcctgaccgccctgcagcaattttacacatgcatcagcgcccctgacggtaccctgaacaaagccgctttcctgttcaagaccctggaaacctgtcagaaagacaagatctacatcagcggcaaggacatcaacagcctgtctaaggccctgtacggcggcagcaactggaacaccctccgggaagccatcctgacagctctgcaaaatgagaaagagtacaaaaagctgctaaaggaaaacgacgaggccgccatcgacagacagctgagcaagaactacttttctctggatttcctgcagacccacgccgacaacctgctgatcaccaaaatcgccgagtacttccagacacagcacgagttagcccaacaagctgtccagaccgatctgctgggaacacaaaacctgaaggccaccgtgaagaagtctctggacgccctgctggactattacagattcattagcctgttcaagaccgatgattccctggacaaagaccctgagttctacggcagctacgaagagatcctgtcccagctcaaggagatcgtgtacatctataacaagaccagaaactacatgactaagaagcccttcaacgaggaaaagttcaagctgaacttcggcaatgcccagctggctggcggctgggacgccaacaaggaggctgattacacatctatcattctgaccaaagatagtgcctactacctgggcatcatgaacaaaaatagcaagcccgacatcgacacagccatcaccgagtccacagataatacctaccggaagatggtgtacaaatacttccccgacttttctaagatgttcgtgaagtgcaccatggtcaaagaagtgaagaatcactttcaggagaacagcacagactttattctgcagaccaacagcttcatcaagcctctgaaaatcaccaaggagatatacgacctgtacagcacagaatacgacggcaagaaaaagttccagattgactacctgcggcgcacaggagatgcccagggctactacgacgccctgaataaatggatcggcttcgccctggccttcatgagaagctacgacagtaccagtatttacgacatcagccagatcgaggctacccagtacgagcggctggataccttttacaaggatatcaacaacatctgctacaacatcagcttcgtgaacctggcagctgataaggtggacaagtgggtcgctgaagggaagctgtacctgtttaagatctataacaaggacttcgcccctggcagaagcggcacaaagaacctgcacaccctgtacctggaaagcatcttcgatgaacagaacctcaaggacgtggtggtgaagctgaacggaggagccgaactgttctacagacggaagaccaagggtaatccctcagtgcactccaagggatctaagctggtgaaccgaacaacagccgacggacagacgatcccggaggaaatctaccaggagatctacctgtacgccaacgggaacaaggaagctcttagccagcaggcccaggagtactatcctagagctgtgatcaagaccgccacacacgacatcgtgaaagacagacggtactatgaggacaagtttttcttccacttccccatcaccatcaactacaaggcccctgattccagcggcgctttcaaccaggaggtgctggcctacctgcaccacaacccagacgtcaatatcatcggcatcgatcggggcgagcggaacctgatctacatgaccgtgatcaaccagcagggccagatcctggccagcaagagcttcaactctattagccaacaggccggcacagtgaacaagtccgttgactaccacgataaactggacaagagagagctggaaagaaccgaagccagaaagagctgggagaccattggcaagatcgccaccctgaaggagggctacctgagcgccgtgatccacgagatcgccaaatacatggtgaagtacaatgccatcgtggtgatggaagatctgaatgccggcttcaagcgtatgcggggcgggatcgctgagaaggccgtgtaccagaagttcgagaaaatgctgatcgacaagctgaactacctcgtgttcaagaacctgcctgccaacgagcctggcggagtgctgaagggctaccagctgagcagcaagttcgacagcttcgagaagatgggcaaacagaacggcttcctgttttacgttcctgccgcctttaccagcaaaatcgatcccgtgaccggcttcagttctgtgttcaagttcaaggaatacaccgccaataccgagaaagctaagtccttcttcacaagcttcgatagcatcgtgtactcttccgaaaaggacgtgtttaccttcagcttcgattatgataatttcaagacatatgctaatatgtacagaaagaagtggcaggtgtccagctatggcgaaagaatcaagcacgagaagaacgtgatcggccactacatcgactcttttgtgaaccctacccaggacatcaagaccctgttcgatacccagaatatcgactatagcagcgagcagaacattctgccacagctgctgcagatcgaagataacaaagccagccacagcttctggaagggcatgctgagcgctttccaaagaatcgtgcagctgagaaatagctcacggacactgaccaccgacggcaaggagacggactacatcatctctcccgtggccgactcctctggaagctattttgactctagaaccgctgacagcgctctgcctcaggacgccgatgccaacggagcttatcacatcgccctgaagggcctgatctacctgaatatcaacaacgccgccaaggacaacaagcctaagctgtacgtgaagaacgaggactggttcaagttcacccaggaaagaaacagcgccaag

SEQ ID NO: 5 - RNA, 19 nt, unidentified
  source 1..19: mol_type = mRNA
aatttctactattgtagat

SEQ ID NO: 6 - RNA, 19 nt, unidentified
  source 1..19: mol_type = mRNA
aatttctactgttgtagat

SEQ ID NO: 7 - RNA, 19 nt, unidentified
  source 1..19: mol_type = mRNA
aatttctactcttgtagat

SEQ ID NO: 8 - DNA, 41 bp, synthetic construct
  source 1..41: mol_type = other DNA
ccatttaagagcagacattagtttttcatcgtggtggttat

SEQ ID NO: 9 - DNA, 41 bp, synthetic construct
  source 1..41: mol_type = other DNA
ccatttatgagcagacattagtttttcatcgtggtggttat

SEQ ID NO: 10 - DNA, 41 bp, synthetic construct
  source 1..41: mol_type = other DNA
ccatttaacagcagacattagtttttcatcgtggtggttat

SEQ ID NO: 11 - DNA, 41 bp, synthetic construct
  source 1..41: mol_type = other DNA
ccatttaagtgcagacattagtttttcatcgtggtggttat

SEQ ID NO: 12 - DNA, 41 bp, synthetic construct
  source 1..41: mol_type = other DNA
ccatttaagaccagacattagtttttcatcgtggtggttat

SEQ ID NO: 13 - DNA, 41 bp, synthetic construct
  source 1..41: mol_type = other DNA
ccatttaagaggagacattagtttttcatcgtggtggttat

SEQ ID NO: 14 - DNA, 41 bp, synthetic construct
  source 1..41: mol_type = other DNA
ccatttaagagctgacattagtttttcatcgtggtggttat

SEQ ID NO: 15 - DNA, 41 bp, synthetic construct
  source 1..41: mol_type = other DNA
ccatttaagagcacacattagtttttcatcgtggtggttat

SEQ ID NO: 16 - DNA, 41 bp, synthetic construct
  source 1..41: mol_type = other DNA
ccatttaagagcagtcattagtttttcatcgtggtggttat

SEQ ID NO: 17 - DNA, 41 bp, synthetic construct
  source 1..41: mol_type = other DNA
ccatttaagaccagagattagtttttcatcgtggtggttat

SEQ ID NO: 18 - DNA, 41 bp, synthetic construct
  source 1..41: mol_type = other DNA
ccatttaagaggagactttagtttttcatcgtggtggttat

SEQ ID NO: 19 - DNA, 41 bp, synthetic construct
  source 1..41: mol_type = other DNA
ccatttaagagcagacaatagtttttcatcgtggtggttat

SEQ ID NO: 20 - DNA, 41 bp, synthetic construct
  source 1..41: mol_type = other DNA
ccatttaagaggagacataagtttttcatcgtggtggttat

SEQ ID NO: 21 - DNA, 41 bp, synthetic construct
  source 1..41: mol_type = other DNA
ccatttaagagcagacatttgtttttcatcgtggtggttat

SEQ ID NO: 22 - DNA, 41 bp, synthetic construct
  source 1..41: mol_type = other DNA
ccatttaagagcagacattactttttcatcgtggtggttat

SEQ ID NO: 23 - DNA, 41 bp, synthetic construct
  source 1..41: mol_type = other DNA
ccatttaagagcagacattagattttcatcgtggtggttat

SEQ ID NO: 24 - DNA, 41 bp, synthetic construct
  source 1..41: mol_type = other DNA
ccatttaagagcagacattagtatttcatcgtggtggttat

SEQ ID NO: 25 - DNA, 41 bp, synthetic construct
  source 1..41: mol_type = other DNA
ccatttaagagcagacattagttattcatcgtggtggttat

SEQ ID NO: 26 - DNA, 41 bp, synthetic construct
  source 1..41: mol_type = other DNA
ccatttaagagcagacattagtttatcatcgtggtggttat

SEQ ID NO: 27 - DNA, 41 bp, synthetic construct
  source 1..41: mol_type = other DNA
ccatttaagagcagacattagttttacatcgtggtggttat

SEQ ID NO: 28 - DNA, 41 bp, synthetic construct
  source 1..41: mol_type = other DNA
ccatttaagagcagacattagtttttgatcgtggtggttat

SEQ ID NO: 29 - DNA, 752 bp, African swine fever virus
  source 1..752: mol_type = genomic DNA
ctgtaacgcagcacagctgaaccgttctgaagaagaagaaagttaatagcagatgccgataccacaagatcagccgtagtgatagaccccacgtaatccgtgtcccaactaatataaaattctcttgctctggatacgttaatatgaccactgggttggtattcctcccgtggcttcaaagcaaaggtaatcatcatcgcacccggatcatcgggggttttaatcgcattgcctccgtagtggaagggtatgtaagagctgcagaactttgatggaaatttatcgataagattgataccatgagcagttacggaaatgtttttaataataggtaatgtgatcggatacgtaacggggctaatatcagatatagatgaacatgcgtctggaagagctgtatctctatcctgaaagcttatctctgcgtggtgagtgggctgcataatggcgttaacaacatgtccgaacttgtgccaatctcggtgttgatgaggattttgatcggagatgttccaggtaggttttaatcctataaacatatattcaatgggccatttaagagcagacattagtttttcatcgtggtggttattgttggtgtgggtcacctgcgttttatggacacgtatcagcgaaaagcgaacgcgttttacaaaaaggttgtgtatttcaggggttacaaacaggttattgatgtaaagttcattattcgtgagcgagatttcattaatgactcctgggataaaccatgg

SEQ ID NO: 30 - RNA, 40 nt, synthetic construct
  source 1..40: mol_type = other RNA
aatttctactattgtagattagagcagacattagtttttc

SEQ ID NO: 31 - DNA, 20 bp, synthetic construct
  source 1..20: mol_type = other DNA
ctgtaacgcagcacagctga

SEQ ID NO: 32 - DNA, 20 bp, synthetic construct
  source 1..20: mol_type = other DNA
ccatggtttatcccaggagt

SEQ ID NO: 33 - DNA, 97 bp, unidentified
  source 1..97: mol_type = genomic DNA
ggccagtgaattcgagctcggtacccgggnnnnnnngagaagtcatttaataaggccactgttaaaaagcttggcgtaatcatggtcatagctgttt

SEQ ID NO: 34 - DNA, 21 bp, synthetic construct
  source 1..21: mol_type = other DNA
ggccagtgaattcgagctcgg

SEQ ID NO: 35 - DNA, 27 bp, synthetic construct
  source 1..27: mol_type = other DNA
aaacagctatgaccatgattacgccaa

SEQ ID NO: 36 - RNA, 44 nt, synthetic construct
  source 1..44: mol_type = other RNA
aatttctactattgtagattgagaagtcatttaataaggccact

SEQ ID NO: 37 - DNA, 28 bp, synthetic construct
  source 1..28: mol_type = other DNA
caatttcacacaggaaacagctatgacc

SEQ ID NO: 38 - DNA, 18 bp, synthetic construct
  source 1..18: mol_type = other DNA
catatattcaatgggcca

SEQ ID NO: 39 - DNA, 17 bp, synthetic construct
  source 1..17: mol_type = other DNA
atcgtggtggttattgt

SEQ ID NO: 40 - DNA, 18 bp, synthetic construct
  source 1..18: mol_type = other DNA
tttcgctgatacgtgtcc

SEQ ID NO: 41 - DNA, 59 bp, synthetic construct
  source 1..59: mol_type = other DNA
catatattcaatgggccaaataagagcagacattagtttttcatcgtggtggttattgt

SEQ ID NO: 42 - DNA, 59 bp, synthetic construct
  source 1..59: mol_type = other DNA
catatattcaatgggccaaattagagcagacattagtttttcatcgtggtggttattgt

SEQ ID NO: 43 - DNA, 59 bp, synthetic construct
  source 1..59: mol_type = other DNA
catatattcaatgggccaaatgagagcagacattagtttttcatcgtggtggttattgt

SEQ ID NO: 44 - DNA, 59 bp, synthetic construct
  source 1..59: mol_type = other DNA
catatattcaatgggccaaatcagagcagacattagtttttcatcgtggtggttattgt

SEQ ID NO: 45 - DNA, 59 bp, synthetic construct
  source 1..59: mol_type = other DNA
catatattcaatgggccaagtaagagcagacattagtttttcatcgtggtggttattgt

SEQ ID NO: 46 - DNA, 59 bp, synthetic construct
  source 1..59: mol_type = other DNA
catatattcaatgggccaagttagagcagacattagtttttcatcgtggtggttattgt

SEQ ID NO: 47 - DNA, 59 bp, synthetic construct
  source 1..59: mol_type = other DNA
catatattcaatgggccaagtgagagcagacattagtttttcatcgtggtggttattgt

SEQ ID NO: 48 - DNA, 59 bp, synthetic construct
  source 1..59: mol_type = other DNA
catatattcaatgggccaagtcagagcagacattagtttttcatcgtggtggttattgt

SEQ ID NO: 49 - DNA, 59 bp, synthetic construct
  source 1..59: mol_type = other DNA
catatattcaatgggccaactaagagcagacattagtttttcatcgtggtggttattgt

SEQ ID NO: 50 - DNA, 59 bp, synthetic construct
  source 1..59: mol_type = other DNA
catatattcaatgggccaacttagagcagacattagtttttcatcgtggtggttattgt

SEQ ID NO: 51 - DNA, 59 bp, synthetic construct
  source 1..59: mol_type = other DNA
catatattcaatgggccaactgagagcagacattagtttttcatcgtggtggttattgt

SEQ ID NO: 52 - DNA, 59 bp, synthetic construct
  source 1..59: mol_type = other DNA
catatattcaatgggccaactcagagcagacattagtttttcatcgtggtggttattgt

SEQ ID NO: 53 - DNA, 59 bp, synthetic construct
  source 1..59: mol_type = other DNA
catatattcaatgggccaattaagagcagacattagtttttcatcgtggtggttattgt

SEQ ID NO: 54 - DNA, 59 bp, synthetic construct
  source 1..59: mol_type = other DNA
catatattcaatgggccaatttagagcagacattagtttttcatcgtggtggttattgt

SEQ ID NO: 55 - DNA, 59 bp, synthetic construct
  source 1..59: mol_type = other DNA
catatattcaatgggccaattgagagcagacattagtttttcatcgtggtggttattgt

SEQ ID NO: 56 - DNA, 59 bp, synthetic construct
  source 1..59: mol_type = other DNA
catatattcaatgggccaattcagagcagacattagtttttcatcgtggtggttattgt

SEQ ID NO: 57 - DNA, 59 bp, synthetic construct
  source 1..59: mol_type = other DNA
catatattcaatgggccacccaagagcagacattagtttttcatcgtggtggttattgt

SEQ ID NO: 58 - DNA, 59 bp, synthetic construct
  source 1..59: mol_type = other DNA
catatattcaatgggccatttaagagcagacattagtttttcatcgtggtggttattgt

SEQ ID NO: 59 - DNA, 752 bp, African swine fever virus
  source 1..752: mol_type = genomic DNA
ctgtaacgcagcacagctgaaccgttctgaagaagaagaaagttaatagcagatgccgataccacaagatcagccgtagtgatagaccccacgtaatccgtgtcccaactaatataaaattctcttgctctggatacgttaatatgaccactgggttggtattcctcccgtggcttcaaagcaaaggtaatcatcatcgcacccggatcatcgggggttttaatcgcattgcctccgtagtggaagggtatgtaagagctgcagaactttgatggaaatttatcgataagattgataccatgagcagttacggaaatgtttttaataataggtaatgtgatcggatacgtaacggggctaatatcagatatagatgaacatgcgtctggaagagctgtatctctatcctgaaagcttatctctgcgtggtgagtgggctgcataatggcgttaacaacatgtccgaacttgtgccaatctcggtgttgatgaggattttgatcggagatgttccaggtaggttttaatcctataaacatatattcaatgggccannnnagagcagacattagtttttcatcgtggtggttattgttggtgtgggtcacctgcgttttatggacacgtatcagcgaaaagcgaacgcgttttacaaaaaggttgtgtatttcaggggttacaaacaggttattgatgtaaagttcattattcgtgagcgagatttcattaatgactcctgggataaaccatgg

SEQ ID NO: 60 - DNA, 12 bp, synthetic construct
  source 1..12: mol_type = other DNA
gtatccagtgcg

SEQ ID NO: 61 - DNA, 752 bp, African swine fever virus; identical to SEQ ID NO: 29
  source 1..752: mol_type = genomic DNA

SEQ ID NO: 62 - RNA, 40 nt, synthetic construct
  source 1..40: mol_type = other RNA
aatttctactattgtagattctcccgtggcttcaaagcaa

SEQ ID NO: 63 - RNA, 40 nt, synthetic construct
  source 1..40: mol_type = other RNA
aatttctactattgtagattataccatgagcagttacgga

SEQ ID NO: 64 - RNA, 40 nt, synthetic construct
  source 1..40: mol_type = other RNA
aatttctactattgtagattaagccacgggaggaatacca

SEQ ID NO: 65 - RNA, 40 nt, synthetic construct
  source 1..40: mol_type = other RNA
aatttctactattgtagattcactacggaggcaatgcgat

SEQ ID NO: 66 - RNA, 40 nt, synthetic construct
  source 1..40: mol_type = other RNA
aatttctactattgtagattcgtaactgctcatggtatca

SEQ ID NO: 67 - RNA, 40 nt, synthetic construct
  source 1..40: mol_type = other RNA
aatttctactattgtagattaaagcaaaggtaatcatcat

SEQ ID NO: 68 - RNA, 40 nt, synthetic construct
  source 1..40: mol_type = other RNA
aatttctactattgtagattgatggaaatttatcgataag

SEQ ID NO: 69 - RNA, 40 nt, synthetic construct
  source 1..40: mol_type = other RNA
aatttctactattgtagattcatacccttccactacggag

SEQ ID NO: 70 - RNA, 40 nt, synthetic construct
  source 1..40: mol_type = other RNA
aatttctactattgtagattcggaaatgtttttaataata

SEQ ID NO: 71 - RNA, 40 nt, synthetic construct
  source 1..40: mol_type = other RNA
aatttctactattgtagattatctatatctgatattagcc

SEQ ID NO: 72 - RNA, 40 nt, synthetic construct
  source 1..40: mol_type = other RNA
aatttctactattgtagattttaataataggtaatgtgat

SEQ ID NO: 73 - DNA, 141 bp, African swine fever virus
  source 1..141: mol_type = genomic DNA
ccatttaagagcagacattagtttttcatcgtggtggttattgttggtgtgggtcacctgcgttttatggacacgtatcagcgaaaagcgaacgcgttttacaaaaaggttgtgtatttcaggggttacaaacaggttatt

SEQ ID NO: 74 - DNA, 28 bp, synthetic construct
  source 1..28: mol_type = other DNA
ccatttaagagcagacattagtttttca

SEQ ID NO: 75 - DNA, 28 bp, synthetic construct
  source 1..28: mol_type = other DNA
ccaattaagagcagacattagtttttca

SEQ ID NO: 76 - DNA, 28 bp, synthetic construct
  source 1..28: mol_type = other DNA
ccatataagagcagacattagtttttca

SEQ ID NO: 77 - DNA, 28 bp, synthetic construct
  source 1..28: mol_type = other DNA
ccattaaagagcagacattagtttttca

SEQ ID NO: 78 - DNA, 28 bp, synthetic construct
  source 1..28: mol_type = other DNA
ccatttatgagcagacattagtttttca

SEQ ID NO: 79 - DNA, 28 bp, synthetic construct
  source 1..28: mol_type = other DNA
ccatttaacagcagacattagtttttca

SEQ ID NO: 80 - DNA, 28 bp, synthetic construct
  source 1..28: mol_type = other DNA
ccatttaagtgcagacattagtttttca

SEQ ID NO: 81 - DNA, 28 bp, synthetic construct
  source 1..28: mol_type = other DNA
ccatttaagaccagacattagtttttca

SEQ ID NO: 82 - DNA, 28 bp, synthetic construct
  source 1..28: mol_type = other DNA
ccatttaagaggagacattagtttttca

SEQ ID NO: 83 - DNA, 28 bp, synthetic construct
  source 1..28: mol_type = other DNA
ccatttaagagctgacattagtttttca

SEQ ID NO: 84 - DNA, 28 bp, synthetic construct
  source 1..28: mol_type = other DNA
ccatttaagagcacacattagtttttca

SEQ ID NO: 85 - DNA, 28 bp, synthetic construct
  source 1..28: mol_type = other DNA
ccatttaagagcagtcattagtttttca

SEQ ID NO: 86 - DNA, 28 bp, synthetic construct
  source 1..28: mol_type = other DNA
ccatttaagagcagagattagtttttca

SEQ ID NO: 87 - DNA, 28 bp, synthetic construct
  source 1..28: mol_type = other DNA
ccatttaagagcagactttagtttttca

SEQ ID NO: 88 - DNA, 36 bp, synthetic construct
  source 1..36: mol_type = other DNA
ccatttaagagcagacaatagtttttcatcgtggtg

SEQ ID NO: 89 - DNA, 36 bp, synthetic construct
  source 1..36: mol_type = other DNA
ccatttaagagcagacataagtttttcatcgtggtg

SEQ ID NO: 90 - DNA, 36 bp, synthetic construct
  source 1..36: mol_type = other DNA
ccatttaagagcagacatttgtttttcatcgtggtg

SEQ ID NO: 91 - DNA, 36 bp, synthetic construct
  source 1..36: mol_type = other DNA
ccatttaagagcagacattactttttcatcgtggtg

SEQ ID NO: 92 - DNA, 36 bp, synthetic construct
  source 1..36: mol_type = other DNA
ccatttaagagcagacattagattttcatcgtggtg

SEQ ID NO: 93 - DNA, 36 bp, synthetic construct
  source 1..36: mol_type = other DNA
ccatttaagagcagacattagtatttcatcgtggtg

SEQ ID NO: 94 - DNA, 36 bp, synthetic construct
  source 1..36: mol_type = other DNA
ccatttaagagcagacattagttattcatcgtggtg

SEQ ID NO: 95 - DNA, 36 bp, synthetic construct
  source 1..36: mol_type = other DNA
ccatttaagagcagacattagtttatcatcgtggtg

SEQ ID NO: 96 - DNA, 36 bp, synthetic construct
  source 1..36: mol_type = other DNA
ccatttaagagcagacattagttttacatcgtggtg

SEQ ID NO: 97 - DNA, 36 bp, synthetic construct
  source 1..36: mol_type = other DNA
ccatttaagagcagacattagtttttgatcgtggtg

SEQ ID NO: 98 - DNA, 29 bp, synthetic construct
  source 1..29: mol_type = other DNA
caataacctgtttgtaacccctgaaatac

SEQ ID NO: 99 - DNA, 579 bp, unidentified
  source 1..579: mol_type = genomic DNA
gccctacatctgctctccctccactaagaagaacctctttgtgtggcgaaagtaaaagtattagggcttttaagttgcccagagtcaaggaacacggataaagacgctgggagattgacatgcatttcgaccaatagcattgcagagaggcgtatcatttcgcggatgttccaatcagtacgcagagagtcgccgtctccaaggtgaaagcggaagtagggccttcgcgcacctcatggaatcccttctgcagcacctggatcgcttttccgagcttctggcggtctcaagcactacctacgtcagcacctgggaccccgccaccgtgcgccgggccttgcagtgggcgcgctacctgcgccacatccatcggcgctttggtcggcatggccccattcgcacggctctggagcggcggctgcacaaccagtggaggcaagagggcggctttgggcggggtccagttccgggattagcgaacttccaggccctcggtcactgtgacgtcctgctctctctgcgcctgctggagaaccgggccctcggggatgcagctcgttaccacctggtgcagcaact

SEQ ID NO: 100 - RNA, 40 nt, synthetic construct
  source 1..40: mol_type = other RNA
aatttctactattgtagattgtcggcatggccccattcgc

SEQ ID NO: 101 - DNA, 591 bp, unidentified
  source 1..591: mol_type = genomic DNA
gccctacatctgctctccctccactaagaagaacctctttgtgtggcgaaagtaaaagtattagggcttttaagttgcccagagtcaaggaacacggataaagacgctgggagattgacatgcatttcgaccaatagcattgcagagaggcgtatcatttcgcggatgttccaatcagtacgcagagagtcgccgtctccaaggtgaaagcggaagtagggccttcgcgcacctcatggaatcccttctgcagcacctggatcgcttttccgagcttctggcggtctcaagcactacctacgtcagcacctgggaccccgccaccgtgcgccgggccttgcagtgggcgcgctacctgcgccacatccatcggcgctttggtcggcatggccccattcgcacggctctggagcggcggctgcacaaccagtggaggcaagagggcggctttgggcggggtccagttccgggattagcgaacttccaggccctcggtcactgtgacgtcctgctctctctgcgcctgctggagaaccgggccctcggggatgcagctcgttaccacctggtgcagcaactctttcccggccc

SEQ ID NO: 102 - DNA, 556 bp, unidentified
  source 1..556: mol_type = genomic DNA
catcaccaacccacagccaaggcggcgctggctttttttttttttttaatctttaacaatttgaatatttgtttttacaaaggtgcattttttaatagggcttggggagtcccagaggtatccagcagaggggagaagaaagagagatgtagggctagaggggtgaggctgaaacagtgacctgtcttggttttcgctccgaaggtaaaagaaatcattgagtcccccgccttcagaagagggtgcattttcaggaggaagcgatggcttcagacagcatatttgagtcatttccttcgtacccacagtgcttcatgagaggtgagtacatgctggtcttgtaatatctacttttgctcagctttgcctgtaatgaaatggcagcttgtttcacctcggtgcagagatgcctcggtgcctgccagttccctgtcttgtttgtgagaggaattcaaactgaggcatatgattacaagtctattggattacttactaatcagatggaagctcttcagaaatgttttaataaatacttagttatgctgttggagtgttc

SEQ ID NO: 103 - DNA, 694 bp, unidentified
  source 1..694: mol_type = genomic DNA
ggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctcccttttgttttgatgcatttctgttttaatttattttccaggcaccactgtagtttagtgatccccagtgtcccccttccctatgg

SEQ ID NO: 104 - RNA, 40 nt, synthetic construct
  source 1..40: mol_type = other RNA
aatttctactattgtagatttggttgcccaccctagtcat

SEQ ID NO: 105 - RNA, 40 nt, synthetic construct
  source 1..40: mol_type = other RNA
aatttctactattgtagatttactttgtcctccggttctg

(19) United States
(12) Patent Application Publication
(10) Pub. No.: US 2024/0254465 A1
(43) Pub. Date: Aug. 1, 2024

(54) HEAT-RESISTANT ENDONUCLEASE AND GENE EDITING SYSTEM MEDIATED BY HEAT-RESISTANT ENDONUCLEASE

(71) Applicant: HUAZHONG AGRICULTURAL UNIVERSITY, Wuhan (CN)

(72) Inventors: Shengsong XIE, Wuhan (CN); Shuhong ZHAO, Wuhan (CN); Xinyun LI, Wuhan (CN); Sheng LI, Wuhan (CN); Bingrong XU, Wuhan (CN); Dagang TAO, Wuhan (CN); Liangliang FU, Wuhan (CN); Jinxue RUAN, Wuhan (CN); Heng WANG, Wuhan (CN); Yunlong MA, Wuhan (CN)

(73) Assignee: HUAZHONG AGRICULTURAL UNIVERSITY, Wuhan (CN)

(21) Appl. No.: 18/605,895

(22) Filed: Mar. 15, 2024

(30) Foreign Application Priority Data
Jan. 17, 2023 (CN) 202310086152.7

(51) Int. Cl. C12N 9/22; C12N 15/11; C12N 15/90

(52) U.S. Cl. CPC C12N 9/22; C12N 15/11; C12N 15/907; C12N 2310/20; C12N 2800/80

(57) ABSTRACT

A nucleic acid endonuclease with high activity and high heat resistance and a gene editing system mediated by the nucleic acid endonuclease are provided. Specifically, the present invention provides a nucleic acid endonuclease Gs12-7 with a wide temperature range identified by metagenomics combined with experiments, which has the advantages of high protein temperature tolerance, recognition of PAM sequences containing BTYV, and thus has a larger gene editing space and high activity and specificity in cleaving target DNA in the genome. The present invention establishes a nucleic acid visualization detection and genome targeted editing technology mediated by the CRISPR/Gs12-7 system, which has broad application prospects in the field of genome targeted modification and nucleic acid detection.

### CROSS REFERENCE TO THE REPLATED APPLICATIONS

This application is based upon and claims priority to Chinese Patent Application No. 202310086152.7, filed on Jan. 17, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBWHYC006_Sequence_Listing.xml, created on 03/12/2024, and is 112,377 bytes in size.

### TECHNICAL FIELD

The present invention relates to the field of genome editing technology, specifically involving the development and application of newly identified guide RNA-mediated heat-resistant endonuclease Gs12-7 and nucleic acid detection, as well as genome targeted editing technology mediated by the newly identified guide RNA-mediated heat-resistant endonuclease Gs12-7.

### BACKGROUND

The gene editing technology mediated by a clustered regularly interspaced short palindromic repeats (CRISPR)/CRISPR associated (Cas) system has become popular worldwide after nearly 10 years of development, becoming one of the most efficient, simple, cost-effective, and easy-to-operate technologies in existing gene editing and genome modification. This technology has shown infinite potential in basic research, clinical transformation, and agricultural production. The CRISPR/Cas system is a natural immune system in prokaryotes, which comprises two parts: CRISPR locus and Cas gene (CRISPR associated gene). At present, CRISPR/Cas systems can be divided into two categories. The first category is that their effector factors for cleaving exogenous nucleic acids are complexes formed by multiple Cas proteins, including type I, type III, and type IV Cas proteins; The second category: their action factors are relatively single Cas proteins, such as type II Cas9 protein and type V Cas12a protein.

The CRISPR/Cas9 or Cas12a system is mainly composed of Cas9 protein or Cas12a protein and guide RNA (sgRNA or crRNA). Among them, crRNA provides sequence specificity, targeting the paired DNA sequence, thereby providing precise localization for Cas9 nuclease or Cas12a nuclease and ultimately cleaving DNA, thereby achieving gene editing. In addition to crRNA, CRISPR/Cas9 or Cas12a also relies on recognizing the sequences of protospacer adjacent motifs (PAM) on the target DNA when performing editing functions. At present, the most widely used CRISPR system is type II CRISPR/Cas system. In addition to CRISPR/Cas9, there are also CRISPR/Cas12, CRISPR/Cas13, and CRISPR/Cas14. Among them, the PAM sequence recognized by SpCas9 nuclease is “NGG”, while the PAM sequence recognized by Cas12a nuclease is “TTTV or TTV”. The complexity of the PAM sequence determines the upper limit of editable sites. In practical applications, the lack of PAM sequence at the target site often leads to the inability of Cas9 or Cas12a to target, thereby hindering the effectiveness of gene editing. Secondly, gene editing requires consideration of different reaction temperatures in order to be compatible with LAMP or RPA isothermal nucleic acid amplification reactions. Therefore, exploring nucleases with less PAM restriction and high heat resistance has become a research hotspot.

For a long time, researchers have been committed to optimizing and engineering Cas9 or Cas12 proteins to expand their compatibility and heat resistance to different PAM sequences, especially allowing Cas proteins to have broader editing capabilities. Taking SpCas9 as an example, the SpCas9 VRQR mutant that can recognize NGA and the SpCas9 VRER mutant that can recognize NGCG were obtained through the error-prone PCR strategy. The xCas9 3.7 variant that can recognize NGG, NG, GAA, and GAT has been constructed by using directed evolution technology PACE; In addition, a more active SpCas9 NG variant has been developed, and its recognized PAM sequence has been extended to NG. A series of SpCas9 mutants were constructed using PACE technology, which extended the recognized PAM sequences to NRNH (R is A/G, H is A/C/T). These works have almost freed SpCas9 and its mutants from PAM troubles. The SpCas9 protein was modified to develop SpRY whose recognized PAM sequences covering NRN and NYN (Y is C/T) (NRN>NYN). The newly identified Cas12b protein is heat-resistant and only recognizes the PAM sequence of 5′-TTN. However, there is currently no Cas12a nuclease with strong heat resistance and less PAM restriction.

Compared with Cas9, Cas12a has many advantages, such as shorter crRNA, making it easier to be delivered to cells; After cleaving, cohesive ends are generated, which is more conducive to precise genome recognition and editing; The distance between the cleaving site and its recognition site is relatively far, which can achieve the purpose of continuous multiple edits. In addition, the greatest feature of the Cas12a protein lies in that it is not only used for gene editing at the cellular or individual level, but also widely used for highly sensitive and specific detection of small molecules such as nucleic acids or proteins. After binding to the target DNA, Cas12a cleaves the cis-target DNA and the trans-non-target single stranded DNA (ssDNA). If fluorescence and quenching group modified ssDNA are provided as reporter genes during nucleic acid cleavage in vitro, it can be used to indicate the presence of target nucleic acid target molecules. This strategy has been widely used for on-site visualization detection of nucleic acid. At present, there are relatively few known Cas12a proteins, such as natural AsCas12a, LbCas12a, and FnCas12a, as well as artificially modified enhanced enAsCas12a, whose recognized PAM sequences are all “TTTV or TTV”, resulting in a small target recognition range. Although studies have shown that there are differences in the PAM sequences of Cas9 or Cas12a proteins from different bacterial sources, the existence of Cas12a proteins with high heat resistance and few base restrictions in the PAM sequence has not been reported yet.

Therefore, there is still an urgent need in this field to search for CRISPR/Cas12a gene editing systems with high temperature resistance and a wider target recognition range.

### SUMMARY

The present invention has developed for the first time a CRISPR/Gs12-7 gene editing system with high activity and high heat resistance, which has the advantages of high protein temperature tolerance, recognition of PAM sequences containing BTYV, and thus has a larger gene editing space and high activity and specificity in cleaving target DNA in the genome. The present invention also establishes a nucleic acid visualization detection and genome targeted editing technology mediated by Gs12-7 protein.

In order to achieve the objectives as above, the present invention comprises the following technical solutions:

The endonucleases in the CRISPR/Cas system comprise the following proteins:
I. The Gs12-7 protein of the amino acid sequence shown in SEQ ID NO: 1;
II. A protein with more than 80% sequence similarity compared with the amino acid sequence shown in SEQ ID NO: 1, and basically retaining the biological function of the sequence from which it derives;
III. A protein with one or more amino acid substitutions, deletions, or additions compared with the amino acid sequence shown in SEQ ID NO: 1, and basically retaining the biological function of the sequence from which it derives.

Fusion proteins, comprising the endonucleases as above and peptides connected to the N-terminus or C-terminus of the proteins.

Polynucleotides, which are polynucleotides encoding the endonucleases or fusion proteins as above. Vectors or host cells comprising the polynucleotides.

The application of the endonucleases as above in gene editing includes modifying genes, knocking out genes, altering the expression of gene products, repairing mutations, or inserting polynucleotides in prokaryotic genome, eukaryotic genome, or in vitro genes.

A CRISPR/Cas gene editing system comprising the endonucleases, fusion proteins, polynucleotides, vectors, or host cells as above. Furthermore, it also comprises direct repeat sequences that can bind to the endonucleases as above and guiding sequences that can target the target sequence.

A visual nucleic acid detection kit comprising the endonucleases as above, single stranded DNA fluorescence quenching reporter genes, and guide RNA paired with target nucleic acids.

The technical solution of the present invention has the following main beneficial effects:
1. The present invention provides for the first time a novel member of the CRISPR/Cas12a system family, Gs12-7, discovered by combining metagenomics and experimental methods.
2. The present invention discovers a CRISPR/Gs12-7 gene editing system with high activity and high temperature tolerance, which has a larger temperature range of gene editing space and high activity and specificity in cleaving target DNA in the genome.
3. The present invention provides for the first time a nucleic acid visualization detection and genome targeted editing technology mediated by the CRISPR/Gs12-7 system.

### BRIEF DESCRIPTION OF THE DRAWINGS

FIG. 1. Prediction of guide RNA dependent endonuclease Gs12-7 using metagenomic methods and phylogenetic tree analysis.

FIGS. 2A-2B. DR sequence pattern diagram of endonuclease Gs12-7 locus, domain, and guide RNA. FIG. 2A. Schematic diagram of the Gs12-7 locus; FIG. 2B. The secondary structure folding and multiple sequence alignment of the DR sequence of the guide RNA, Gs12-7: SEQ ID NO: 5, LbCas12a and FnCas12a: SEQ ID NO: 6, AsCas12a: SEQ ID NO: 7.

FIG. 3. Conservative analysis of predicted amino acid sequences of Gs12-7 protein and known amino acid sequences of Cas12a proteins (AsCas12a, LbCas12a, and FnCas12a).

FIG. 4. Detection of the activity of Gs12-7 cleaving double stranded DNA target by gel electrophoresis. The target is the amplified fragment of ASFV p72 gene of African swine fever virus, and the recognized target site PAM is “TTTA”.

FIGS. 5A-5B. Identification of the characteristics of recognition of PAM by Gs12-7 using PAM library subtraction experiment in bacteria. The endonuclease recognizes the PAM motif as BTYV (B=G/T/C; Y=C/T; V=G/A/C).

FIGS. 6A-6B. Validation of in vitro cleavage ability of Gs12-7 towards the same target site containing different PAMs in linear double stranded DNA. The target is an amplified fragment of the ASFV p72 gene of African swine fever virus, with the same spacer sequence but different PAM sequences.

FIGS. 7A-7C. Comparison of the trans-cleavage activity of Gs12-7 and wild-type LbCas12a towards ssDNA-FQ reporting system base preference. The target is the amplified fragment of ASFV p72 gene of African swine fever virus, and the recognized target site PAM is “TTTA”. FIG. 7A. Blue light instrument detection results; FIG. 7B and FIG. 7C. Microplate reader (EnSpire Multimode Plate Reader, PerkinElmer) results.

FIG. 8. The optimal enzyme digestion temperature for evaluating the trans-cleavage activity of Gs12-7. The target is the ASFV p72 gene.

FIGS. 9A-9B. Validation of the trans-cleavage activity of Gs12-7 on target sites containing different PAMs in linear double stranded DNA. The target is the amplified fragment of the ASFV p72 gene of African swine fever virus. FIG. 9A. Experimental procedure diagram, FIG. 9B. Blue light instrument detection results.

FIGS. 10A-10B. Evaluation of the positional effect of single base mismatch on the Gs12-7 trans-cleavage activity in the target (“TTTA” and “Target Sequences 1-20” are shown in SEQ ID NO: 8-SEQ ID NO: 28. The target is the amplified fragment of the ASFV p72 gene of African swine fever virus, with TTTA as the positive control.

FIGS. 11A-11B. Detection of genome editing activity of RNP delivered Gs12-7 protein and in vitro transcribed crRNA complex in cells through T7EN1 enzyme digestion assay. The target is the human FANCF gene, and Control is a negative control.

FIGS. 12A-12B. Detection of genome editing activity of single or tandem crRNA expression vectors co-transfected with liposomes into Gs12-7 eukaryotic expression vector in cells through T7EN1 enzyme digestion assay. FIG. 12A. Schematic diagram of a single or tandem crRNA expression vector. FIG. 12B. T7EN1 enzyme digestion experiment. The cell is human HEK293T.

FIGS. 13A-13B. Evaluation of CRISPR/Gs12-7 system mediated multiple gene editing activity in eukaryotic cells. FIG. 13A. Pattern diagram of tandem crRNA expression vector; FIG. 13B. T7EN1 enzyme digestion experiment. The cell is human HEK293T.

### DETAILED DESCRIPTION OF THE EMBODIMENTS

### Terminology

Unless defined otherwise, all technical and scientific terms used herein have the same meaning as commonly understood by one of ordinary skill in the art to which this invention belongs.

Genie scissor (Lingjian) is a family of endonucleases, where Genie means elf, representing bacterial origin, and scissor represents gene scissors, indicating their potential gene editing functions. The Chinese name corresponding to Genie scissor endonuclease is “Lingjian” endonuclease, and the Genie scissor gene editing system represents the gene editing system mediated by “Lingjian” endonuclease, abbreviated as “Lingjian gene editing”.

The protospacer adjacent motif (PAM) is a short DNA sequence (usually the length of 2-6 base pairs). The traditional view is that PAM is necessary for Cas endonuclease cleavage, typically 3-4 nucleotides downstream of the cleavage site. There are many different Cas endonucleases that can be purified from different bacteria, and each enzyme may recognize different PAM sequences.

The present invention will be further described in combination with specific embodiments. It should be understood that these embodiments are only used to illustrate the invention and not to limit the scope of the invention. The following experimental methods without specific conditions are usually in accordance with conventional conditions, such as those described in the Molecular Cloning: Laboratory Manual (New York: Cold Spring Harbor Laboratory Press, 1989), or the conditions recommended by the manufacturer.

### Example 1. Mining Novel Guide RNA Dependent Endonucleases Based on Metagenomics Methods

Based on the bioinformatics identification process of a novel guide RNA dependent endonuclease constructed by the inventor, a deep mining of bacterial encoded proteins was carried out on the massive metagenomic sequencing data from public databases such as the Non-Redundant Protein Sequence Database (NCBI nr) and the Global Microbial Gene Catalog Database (GMGC). The general analysis process was as follows: for all contig sequences in the target database, use minced software to search and locate the CRISPR array, then use prodigal software to predict CRISPR array adjacent expressed proteins, use CD-hit software to remove redundancy from all predicted proteins, use mega software for protein clustering analysis, and use hmmer software to identify and classify CRISPR-Cas similarity proteins. Finally, a new unknown bacterial protein was obtained, with an amino acid sequence as shown in SEQ ID NO: 1 and a nucleic acid sequence as shown in SEQ ID NO: 2.

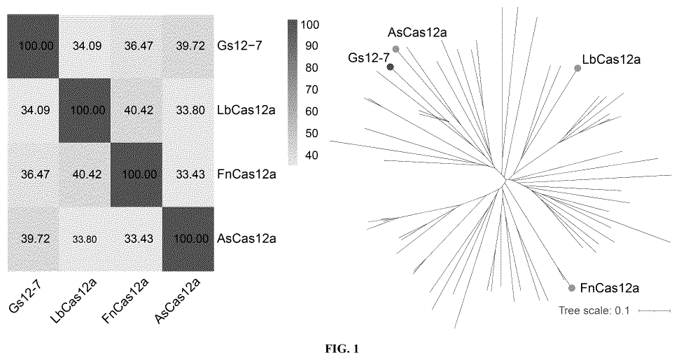

Through phylogenetic tree analysis, it is found that this new bacterial protein is located on different CRISPR-Cas12a phylogenetic branches (FIG. 1), suggesting that it may be a new RNA guided endonuclease. The inventor named the newly discovered proteins from different bacteria as Genie Scissor (GS) endonucleases. For the convenience of subsequent research, based on the origin of bacterial species, the inventor named this new unknown bacterial protein Gs12-7, following the naming convention of “endonuclease+numerical number”.

Next, the inventor utilized a localized blast program to perform sequence similarity alignment between this newly discovered bacterial protein and the NCBI nr database. The results show that the amino acid sequence conservation of the new Gs12-7 protein with known endonucleases LbCas12a, FnCas12a, and AsCas12a are 34.09%, 36.47%, and 39.72%, respectively (FIG. 1).

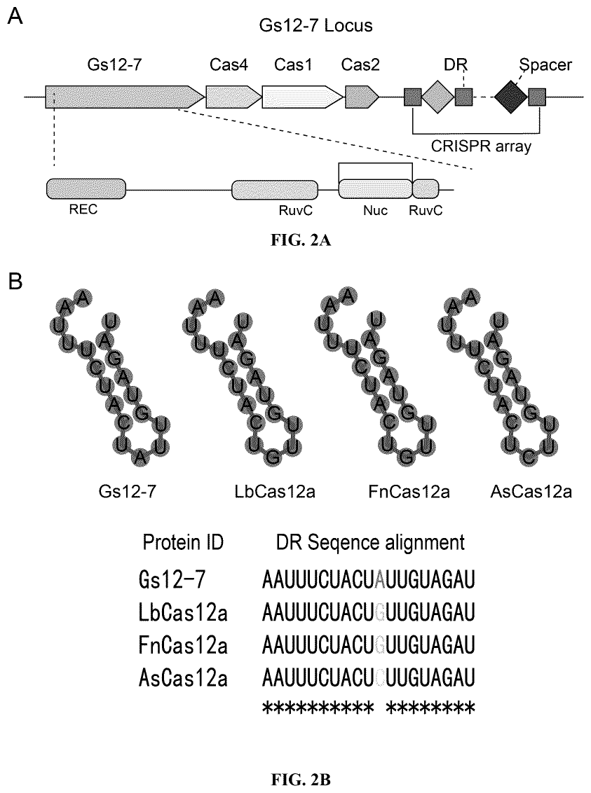

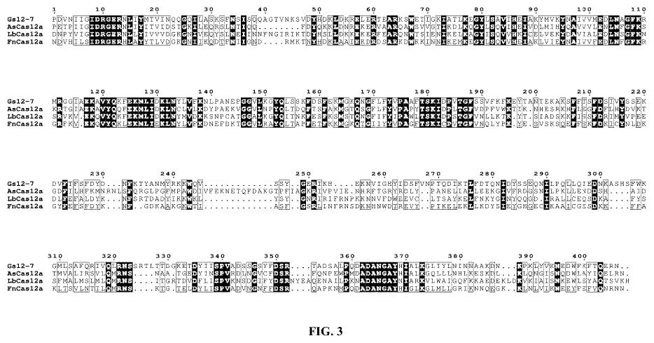

Furthermore, the inventor analyzed the loci of this protein using CRISPRCasFinder software. It is found that Gs12-7 has a CRISPR array sequence, which comprises multiple repeat and interval sequences, as well as Cas4, Cas1, and Cas2 proteins. By using hmmer software to perform hidden Markov model alignment analysis on domain sequences in Pfam database, REC1 domain (Alpha helical recognition lobe domain), RuvC nuclease domain, and NUC domain (Nuclease domain) were obtained, and it is speculated that this new bacterial protein may have nucleic acid cleavage activity; next, the inventor predicted and aligned multiple sequences of the DR sequence secondary structure of Gs12-7 through the RNAfold web server (http://ma.tbi.univie.ac.at/cgi-bin/RNAWebSuite/RNAfold.cgi) online website, and it is found that the newly predicted bacterial protein has a DR secondary structure similar to the known Cas12a protein, but with one base difference (FIGS. 2A-2B).

Finally, the inventor performed amino acid multiple sequence alignment of the RuvC and Nuc domains of Gs12-7 with known LbCas12a, FnCas12a, and AsCas12a proteins, respectively. As shown in FIG. 3, it is found that there is a significant difference in the amino acid sequence similarity between the structure of the Gs12-7 protein and the known Cas12a protein. Therefore, further experiments are urgently needed to determine whether it has nucleic acid directed cleavage activity.

### Example 2. Discovery of In Vitro Nucleic Acid Cleavage Activity of Guide RNA Dependent Gs12-7 Endonuclease

This embodiment tested the cleavage activity of the Gs12-7 protein on double stranded DNA through in vitro experiments. The guide RNA paired with the target nucleic acid was used to guide the recognition and binding of the Gs12-7 protein to the target nucleic acid, thereby stimulating the cleavage activity of the Genie scissor protein towards the target nucleic acid and cleaving the double stranded target nucleic acid in the system. Then, agarose gel electrophoresis was used to observe the size change of the target band to identify its enzyme digestion activity.

In this embodiment, the selected target double stranded DNA (dsDNA) is the African swine fever P72 gene, PAM is TTTA its sequence:

(SEQ ID NO: 29)
CTGTAACGCAGCACAGCTGAACCGTTCTGAAGAAGAAGAAAGTTAATAGC
AGATGCCGATACCACAAGATCAGCCGTAGTGATAGACCCCACGTAATCCG
TGTCCCAACTAATATAAAATTCTCTTGCTCTGGATACGTTAATATGACCA
CTGGGTTGGTATTCCTCCCGTGGCTTCAAAGCAAAGGTAATCATCATCGC
ACCCGGATCATCGGGGGTTTTAATCGCATTGCCTCCGTAGTGGAAGGGTA
TGTAAGAGCTGCAGAACTTTGATGGAAATTTATCGATAAGATTGATACCA
TGAGCAGTTACGGAAATGTTTTTAATAATAGGTAATGTGATCGGATACGT
AACGGGGCTAATATCAGATATAGATGAACATGCGTCTGGAAGAGCTGTAT
CTCTATCCTGAAAGCTTATCTCTGCGTGGTGAGTGGGCTGCATAATGGCG
TTAACAACATGTCCGAACTTGTGCCAATCTCGGTGTTGATGAGGATTTTG
ATCGGAGATGTTCCAGGTAGGTTTTAATCCTATAAACATATATTCAATGG
GCCATTTAAGAGCAGACATTAGTTTTTCATCGTGGTGGTTATTGTTGGTG
TGGGTCACCTGCGTTTTATGGACACGTATCAGCGAAAAGCGAACGCGTTT
TACAAAAAGGTTGTGTATTTCAGGGGTTACAAACAGGTTATTGATGTAAA
GTTCATTATTCGTGAGCGAGATTTCATTAATGACTCCTGGGATAAACCAT
GG;

the bold marking is PAM, and the underline represents the target sequence. The guide RNA sequence is:

(SEQ ID NO: 30, the underlined area is
the target area)
AAUUUCUACUAUUGUAGAUUAGAGCAGACAUUAGUUUUUC.

Using the pmd-18t-p72 plasmid as a template, p72-F: CTGTAACGCAGCACAGCTGA (SEQ ID NO: 31), and p72-R: CCATGGTTTATCCCAGGAGT (SEQ ID NO: 32) as primers, PCR amplification was performed to obtain P72 double stranded DNA. Secondly, the DNA sequence encoding Gs12-7 was synthesized by optimizing the Escherichia coli codon, and NLS nuclear localization signals were added to its C-terminus. The DNA sequence is shown in SEQ ID NO: 3. Subsequently, it was connected to the prokaryotic expression vector pET-28a and transformed into the Escherichia coli BL21 strain. After identifying the positive clone, IPTG induced expression was performed, and the target protein was purified through affinity chromatography. The following system was adopted in the in vitro cleaving reaction: 10×CutSmart Buffer 2 μL. The predicted Geniscissor-NLS-tagged protein was 500 ng, the guide RNA was 500 ng, and the P72 target amplification product was 2 μL. The system was incubated at 37° C. for 0.5 min, 2 min, 10 min, and 20 min, respectively. After the reaction was completed, 1 μL of protease K was added separately and incubated at 55° C. for 10 min to terminate the reaction. Guide RNA and target nucleic acid were added in the experimental group, while guide RNA was not added in the control group. After reaction, 1% agarose gel electrophoresis was carried out, the difference between the newly discovered target bands of Gs12-7 experimental group and control group was detected by UV transilluminator, and the cleaving efficiency was analyzed by Image J software.

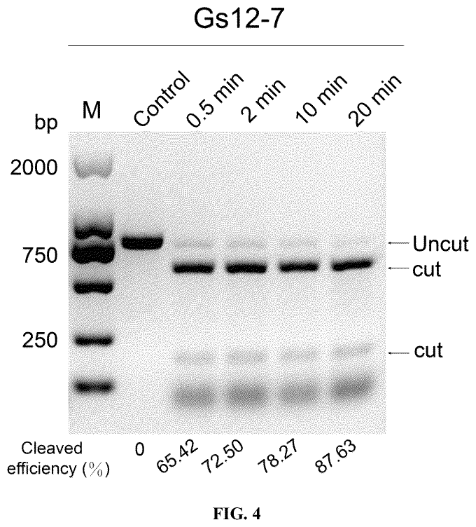

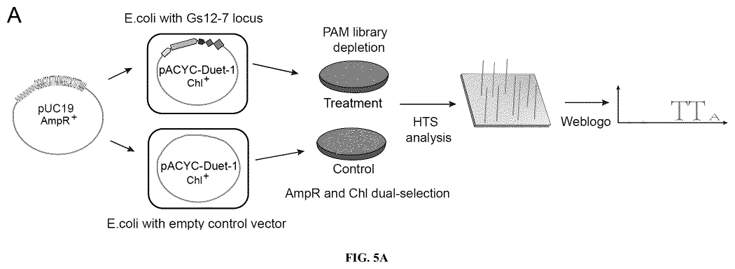

As shown in FIG. 4, compared with the control group without guide RNA, the Gs12-7 protein in the experimental group is able to cleave the target double stranded DNA in just 0.5 min, with two distinct cleavage bands. The cleaving efficiency is calculated to be 65.42%. Especially, it is found that as the reaction time increases, the cleaving efficiency also significantly improves, reaching 72.50%, 78.27%, and 87.63%, respectively. From this, it can be seen that the Gs12-7 protein predicted through metagenomic strategies has a high ability for nucleic acid targeted cleavage.

### Example 3. Discovery of the PAM Motif Specifically Recognized by the CRISPR-Gs12-7 System, BTYV

The PAM sequence recognized by the Gs12-7 protein with low homology and in vitro target nucleic acid cleavage activity was identified through bacterial PAM library subtraction experiment. Among them, the construction process of the randomly mixed PAM vector library is as follows: the DNA oligo sequence

(SEQ ID NO: 33)
GGCCAGTGAATTCGAGCTCGGTACCCGGGNNNNNNNGAGAAGTCATTTAA
TAAGGCCACTGTTAAAAAGCTTGGCGTAATCATGGTCATAGCTGTTT

was synthesized, where Nis a random deoxyribonucleotide. Oligo F: GGCCAGTGAATTCGAGCTCGG (SEQ ID NO: 34) and Oligo R: AAACAGCTATGACCATGATTACGCCAA (SEQ ID NO: 35) were used as upstream and downstream primers for PCR amplification. They were then connected to the pUC19 vector through homologous recombination. After transformation into Escherichia coli, the plasmid was extracted to form a random mixed PAM vector library. The guide RNA sequence used is:

(SEQ ID NO: 36, the underlined area is the
target recognition sequence)
AAUUUCUACUAUUGUAGAUUGAGAAGUCAUUUAAUAAGGCCACU.

Bacterial PAM library subtraction experiment: the constructed vector pACYC-Duet-1-Gs12-7-crRNA co-expressing the predicted Gs12-7 protein and crRNA was transformed into the DE3 (BL21) competent cells to prepare a stable expression bacterial strain. The stable transgenic bacterial strain constructed from the expression vector pACYC-Duet-1-Gs12-7 without crRNA was used as a negative control. 100 ng of PAM library plasmids were transferred into stable expression bacterial strains, and screened using ampicillin and chloramphenicol resistant plates. After 16 hours, the colonies on the plates were scraped off for plasmid extraction. Using 100 ng of extracted plasmids as templates, PCR amplification was performed using library sequencing primers Seq-F: GGCCAGTGAATTCGAGCTCGG (SEQ ID NO: 34) and PAM Seq-R: CAATTTCACACAGGAAACAGCTATGACC (SEQ ID NO: 37). After product recovery, the experimental and control groups were subjected to second-generation high-throughput sequencing, and the sequencing results were analyzed and displayed using Weblogo 3.0.

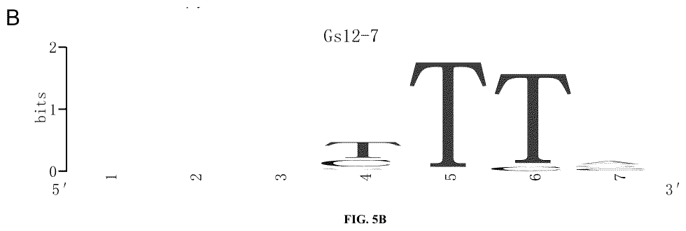

Identification of PAM sequence characteristics recognized by the Gs12-7 protein: 16384 different types of PAM sequences contained in the starting vector library were counted for their frequency of occurrence in high-throughput sequencing in the experimental group and control group, and standardized using the total number of PAM sequences in each group. The calculation method for each PAM consumption change is log 2 (control group standardized value/experimental group standardized value). When this value is greater than 3.5, it is considered that this PAM has been significantly consumed. Then, Weblogo 3.0 was used to visually display the frequency of occurrence of significantly consumed PAM sequences at various positions. As shown in FIGS. 5A-5B, it is found that the Gs12-7 protein recognizes the PAM sequence as BTYV (B=G/T/C; Y=C/T; V=G/A/C), which is different from the reported Cas12a protein specific recognition of PAM as the “TTTV” base composition sequence.

To demonstrate the reliability of the “BTYV” validated by the bacterial PAM library subtraction experiment, it was validated through in vitro enzyme digestion of double stranded DNA. Using the pmd-18t-p72 plasmid as a template, P72 fragment 1 was amplified using P72-F1 and P72-R1 as primers. P72 fragment 2 was amplified using different P72-F2 and P72-R2 primers, and P72 fragment 3 was amplified using P72-F3 and P72-R3 primers. Finally, using P72-F1 and P72-R3 primers, fragments 1, 3, and different fragments 2 as templates, Overlap PCR was performed to obtain different PAM target double stranded DNA (dsDNA) African swine fever P72 gene. The primer sequence is shown in the table below:

| Primer name | Sequence (5′-3′) |
|---|---|
| P72-F1 | CTGTAACGCAGCACAGCTGA (SEQ ID NO: |
| | 31) |
| P72-R1 | CATATATTCAATGGGCCA (SEQ ID NO: |
| | 38) |
| P72-F3 | ATCGTGGTGGTTATTGT (SEQ ID NO: 39) |
| P72-R3 | CCATGGTTTATCCCAGGAGT (SEQ ID NO: |
| | 32) |
| P72-R2 | TTTCGCTGATACGTGTCC (SEQ ID NO: |
| | 40) |
| P72-F2-AATA | CATATATTCAATGGGCCAAATAAGAGCAGACAT |
| | TAGTTTTTCATCGTGGTGGTTATTGT (SEQ |
| | ID NO: 41) |
| P72-F2-AATT | CATATATTCAATGGGCCAAATTAGAGCAGACAT |
| | TAGTTTTTCATCGTGGTGGTTATTGT (SEQ |
| | ID NO: 42) |
| P72-F2-AATG | CATATATTCAATGGGCCAAATGAGAGCAGACAT |
| | TAGTTTTTCATCGTGGTGGTTATTGT (SEQ |
| | ID NO: 43) |
| P72-F2-AATC | CATATATTCAATGGGCCAAATCAGAGCAGACAT |
| | TAGTTTTTCATCGTGGTGGTTATTGT (SEQ |
| | ID NO: 44) |
| P72-F2-AGTA | CATATATTCAATGGGCCAAGTAAGAGCAGACAT |
| | TAGTTTTTCATCGTGGTGGTTATTGT (SEQ |
| | ID NO: 45) |
| P72-F2-AGTT | CATATATTCAATGGGCCAAGTTAGAGCAGACAT |
| | TAGTTTTTCATCGTGGTGGTTATTGT (SEQ |
| | ID NO: 46) |
| P72-F2-AGTG | CATATATTCAATGGGCCAAGTGAGAGCAGACAT |
| | TAGTTTTTCATCGTGGTGGTTATTGT (SEQ |
| | ID NO: 47) |
| P72-F2-AGTC | CATATATTCAATGGGCCAAGTCAGAGCAGACAT |
| | TAGTTTTTCATCGTGGTGGTTATTGT (SEQ |
| | ID NO: 48) |
| P72-F2-ACTA | CATATATTCAATGGGCCAACTAAGAGCAGACAT |
| | TAGTTTTTCATCGTGGTGGTTATTGT (SEQ |
| | ID NO: 49) |
| P72-F2-ACTT | CATATATTCAATGGGCCAACTTAGAGCAGACAT |
| | TAGTTTTTCATCGTGGTGGTTATTGT (SEQ |
| | ID NO: 50) |
| P72-F2-ACTG | CATATATTCAATGGGCCAACTGAGAGCAGACAT |
| | TAGTTTTTCATCGTGGTGGTTATTGT (SEQ |
| | ID NO: 51) |
| P72-F2-ACTC | CATATATTCAATGGGCCAACTCAGAGCAGACAT |
| | TAGTTTTTCATCGTGGTGGTTATTGT (SEQ |
| | ID NO: 52) |
| P72-F2-ATTA | CATATATTCAATGGGCCAATTAAGAGCAGACAT |
| | TAGTTTTTCATCGTGGTGGTTATTGT (SEQ |
| | ID NO: 53) |
| P72-F2-ATTT | CATATATTCAATGGGCCAATTTAGAGCAGACAT |
| | TAGTTTTTCATCGTGGTGGTTATTGT (SEQ |
| | ID NO: 54) |
| P72-F2-ATTG | CATATATTCAATGGGCCAATTGAGAGCAGACAT |
| | TAGTTTTTCATCGTGGTGGTTATTGT (SEQ |
| | ID NO: 55) |
| P72-F2-ATTC | CATATATTCAATGGGCCAATTCAGAGCAGACAT |
| | TAGTTTTTCATCGTGGTGGTTATTGT (SEQ |
| | ID NO: 56) |
| P72-F2-AATA | CATATATTCAATGGGCCAAATAAGAGCAGACAT |
| | TAGTTTTTCATCGTGGTGGTTATTGT (SEQ |
| | ID NO: 41) |
| P72-F2-AATT | CATATATTCAATGGGCCAAATTAGAGCAGACAT |
| | TAGTTTTTCATCGTGGTGGTTATTGT (SEQ |
| | ID NO: 42) |
| P72-F2-AATG | CATATATTCAATGGGCCAAATGAGAGCAGACAT |
| | TAGTTTTTCATCGTGGTGGTTATTGT (SEQ |
| | ID NO: 43) |
| P72-F2-AATC | CATATATTCAATGGGCCAAATCAGAGCAGACAT |
| | TAGTTTTTCATCGTGGTGGTTATTGT (SEQ |
| | ID NO: 44) |
| P72-F2-CCCC | CATATATTCAATGGGCCACCCAAGAGCAGACAT |
| | TAGTTTTTCATCGTGGTGGTTATTGT (SEQ |
| | ID NO: 57) |
| P72-F2-TTTA | CATATATTCAATGGGCCATTTAAGAGCAGACAT |
| | TAGTTTTTCATCGTGGTGGTTATTGT (SEQ |
| | ID NO: 58) |

In this embodiment, the selected different PAM target double stranded DNA (dsDNA) is the African swine fever P72 gene, its sequence:

(SEQ ID NO: 59)
CTGTAACGCAGCACAGCTGAACCGTTCTGAAGAAGAAGAAAGTTAATAGC
AGATGCCGATACCACAAGATCAGCCGTAGTGATAGACCCCACGTAATCCG
TGTCCCAACTAATATAAAATTCTCTTGCTCTGGATACGTTAATATGACCA
CTGGGTTGGTATTCCTCCCGTGGCTTCAAAGCAAAGGTAATCATCATCGC
ACCCGGATCATCGGGGGTTTTAATCGCATTGCCTCCGTAGTGGAAGGGTA
TGTAAGAGCTGCAGAACTTTGATGGAAATTTATCGATAAGATTGATACCA
TGAGCAGTTACGGAAATGTTTTTAATAATAGGTAATGTGATCGGATACGT
AACGGGGCTAATATCAGATATAGATGAACATGCGTCTGGAAGAGCTGTAT
CTCTATCCTGAAAGCTTATCTCTGCGTGGTGAGTGGGCTGCATAATGGCG
TTAACAACATGTCCGAACTTGTGCCAATCTCGGTGTTGATGAGGATTTTG
ATCGGAGATGTTCCAGGTAGGTTTTAATCCTATAAACATATATTCAATGG
GCCANNNNAGAGCAGACATTAGTTTTTCATCGTGGTGGTTATTGTTGGTG
TGGGTCACCTGCGTTTTATGGACACGTATCAGCGAAAAGCGAACGCGTTT
TACAAAAAGGTTGTGTATTTCAGGGGTTACAAACAGGTTATTGATGTAAA
GTTCATTATTCGTGAGCGAGATTTCATTAATGACTCCTGGGATAAACCAT
GG;

the bold marking is PAM, and the underline represents the target sequence. For the same guide RNA sequence:

(SEQ ID NO: 30, the underlined area
is the target area).
AAUUUCUACUAUUGUAGAUUAGAGCAGACAUUAGUUUUUC

Secondly, the DNA sequence encoding Gs12-7 was synthesized by optimizing the Escherichia coli codon, and NLS nuclear localization signals were added to its C-terminus. The DNA sequence is shown in SEQ ID NO: 3. Subsequently, it was connected to the prokaryotic expression vector pET-28a and transformed into the Escherichia coli BL21 strain. After identifying the positive clone, IPTG induced expression was performed, and the target protein was purified through affinity chromatography. The following system was adopted in the in vitro cleaving reaction: 10×CutSmart Buffer 2 μL. The predicted Genie scissor-NLS-tagged protein was 500 ng, the guide RNA was 500 ng, and the P72 target PCR amplification product for different PAMs was 2 μL. The system was incubated at 37° C. for 30 min, respectively. After the reaction was completed, 1 μL of protease K was added separately and incubated at 55° C. for 10 min to terminate the reaction. Guide RNA and target nucleic acid were added in the experimental group, while guide RNA was not added in the control group. After reaction, 1% agarose gel electrophoresis was carried out, the difference between the target bands of the new endonuclease Gs12-7 predicted under different PAM target sites in the experimental group and the control group was detected by imaging observation under UV transilluminator, and the cleaving efficiency was analyzed through Image J software.

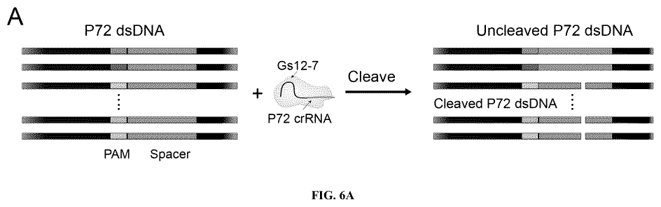

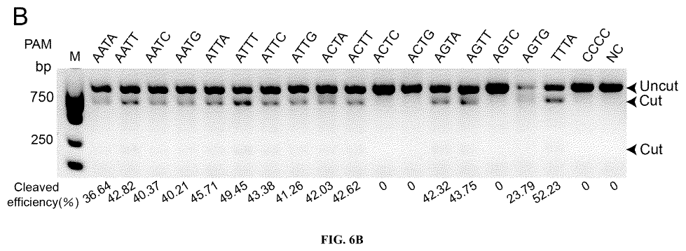

As shown in FIGS. 6A-6B, compared with the control group without guide RNA, for the same crRNA of P72 gene with different PAMs, in the target site of “BTYV” as PAM, the Gs12-7 protein in the experimental group is able to cleave double stranded DNA of different PAMs in the reaction solution. There are two obvious cleavage bands, but the cleaving efficiency is different. In some non-classical PAMs, such as “AATA”, “ATTA”, “ACTA”, “AGTA”, etc., although not as efficient as classical PAMs, there is still a certain degree of cleaving efficiency. However, in other non-classical PAMs such as “ACTC”, “ACTG”, “AGTC”, and “CCCC”, there is no cleaving efficiency, and these non-classical PAMs are worth considering next. From this, it can be seen that the motif of Gs12-7 is identified as “BTYV” through bacterial PAM library subtraction experiment.

### Example 4. On-Site Visualization and Rapid Detection of Nucleic Acids Mediated by CRISPR-Gs12-7 System

Further evaluate whether the Gs12-7 protein has trans-cleavage activity. Guide RNA that could pair with the target nucleic acid was used to guide endonuclease Gs12-7 to recognize and bind to the target nucleic acid; subsequently, its “trans-cleavage” activity towards any single stranded nucleic acid was stimulated, thereby cleaving the single stranded DNA fluorescence quenching reporter gene (ssDNA-FQ) in the reaction system; furthermore, the trans-cleavage function of the Gs12-7 protein could be determined by the excitation fluorescence intensity, background noise, and visual color changes. By screening different base combinations of intermediate single stranded DNA, the optimal fluorescence quenching reporter gene (ssDNA-FQ) for the Gs12-7 protein was identified.

The target double stranded DNA (dsDNA) used in this embodiment is the p72 conserved gene of African swine fever virus ASFV, its sequence:

(SEQ ID NO: 29)
CTGTAACGCAGCACAGCTGAACCGTTCTGAAGAAGAAGAAAGTTAATAGC
AGATGCCGATACCACAAGATCAGCCGTAGTGATAGACCCCACGTAATCCG
TGTCCCAACTAATATAAAATTCTCTTGCTCTGGATACGTTAATATGACCA
CTGGGTTGGTATTCCTCCCGTGGCTTCAAAGCAAAGGTAATCATCATCGC
ACCCGGATCATCGGGGGTTTTAATCGCATTGCCTCCGTAGTGGAAGGGTA
TGTAAGAGCTGCAGAACTTTGATGGAAATTTATCGATAAGATTGATACCA
TGAGCAGTTACGGAAATGTTTTTAATAATAGGTAATGTGATCGGATACGT
AACGGGGCTAATATCAGATATAGATGAACATGCGTCTGGAAGAGCTGTAT
CTCTATCCTGAAAGCTTATCTCTGCGTGGTGAGTGGGCTGCATAATGGCG
TTAACAACATGTCCGAACTTGTGCCAATCTCGGTGTTGATGAGGATTTTG
ATCGGAGATGTTCCAGGTAGGTTTTAATCCTATAAACATATATTCAATGG
GCCATTTAAGAGCAGACATTAGTTTTTCATCGTGGTGGTTATTGTTGGTG
TGGGTCACCTGCGTTTTATGGACACGTATCAGCGAAAAGCGAACGCGTTT
TACAAAAAGGTTGTGTATTTCAGGGGTTACAAACAGGTTATTGATGTAAA
GTTCATTATTCGTGAGCGAGATTTCATTAATGACTCCTGGGATAAACCAT
GG;

the bold marking is PAM, and the underline represents the target sequence. The guide RNA sequence is:

(SEQ ID NO: 30, the underlined area
is the target region)
AAUUUCUACUAUUGUAGAUUAGAGCAGACAUUAGUUUUUC.

The single stranded DNA fluorescence quenching reporter gene sequences are ROX-TATAT-BHQ 2, ROX-TTTTT-BHQ 2, ROX-GGGGG-BHQ 2, ROX-CCCCC-BHQ 2, ROX-AAAAA-BHQ 2, ROX-GCGCG-BHQ 2, or ROX-random-BHQ 2 (5′ROX/GTATCCAGTGCG/3′BHQ 2 (SEQ ID NO: 60)). Firstly, the Gs12-7 and LbCas12a proteins were obtained by prokaryotic expression purification, guide RNA was obtained by in vitro transcription, and the p72 target gene double stranded DNA was obtained by PCR amplification. Next, the following reaction system was adopted: Gs12-7/LbCas12a protein 500 ng, guide RNA 500 ng, 2 μL of 10×CutSmart Buffer, 1 μM of single stranded DNA fluorescence quenching reporter genes with different base combinations, and 2 μL of PCR amplification target product. The negative control was without a target. The reaction was carried at 37° C. for 15 min, and 98° C. for 2 min to inactivate. Then, the preference of Gs12-7 protein trans-cleavage activity for reporter gene bases was detected using a microplate reader and a blue light instrument.

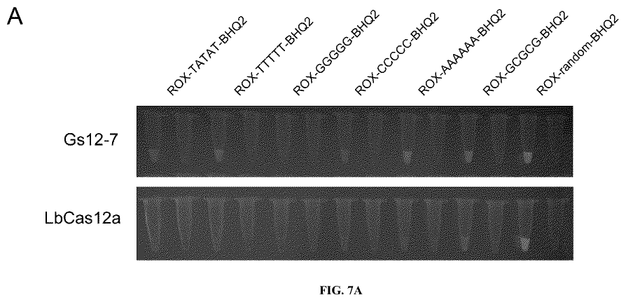

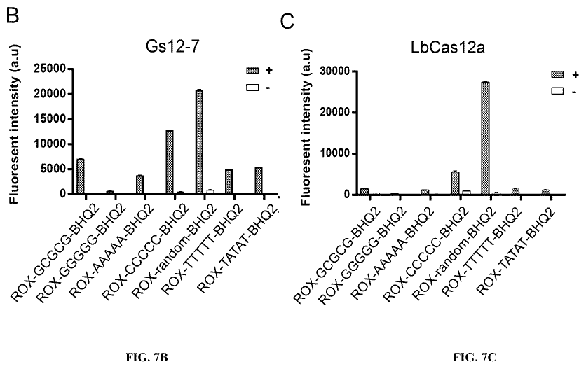

As shown in FIGS. 7A-7C, from the fluorescence changes of the reaction solution before and after cleavage, it can be seen that the newly discovered Gs12-7 protein and the known LbCas12a protein both have nucleic acid trans-cleavage activity; compared with the known LbCas12a, the activated newly identified protein can not only transcleave ROX-GCGCG-BHQ 2 and ROX-random-BHQ 2, but also cleave ROX-TATAT-BHQ 2, ROX-TTTTT-BHQ 2, ROX-CCCCC-BHQ 2, and ROX-AAAAA-BHQ 2 reporter genes. From this, it can be seen that the base composition range of the new Gs12-7 protein trans-cleavage targeted reporter gene is wide and its activity is high.

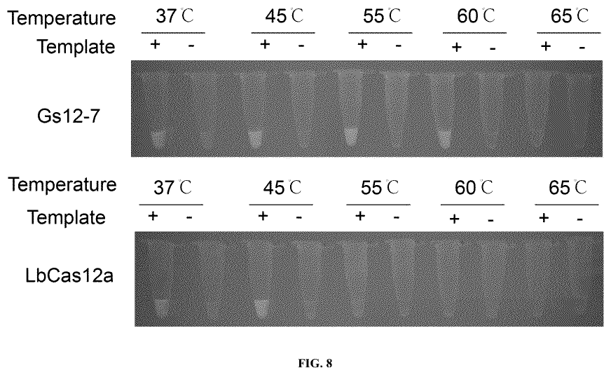

Subsequently, the optimal enzyme digestion reaction temperature for the nucleic acid detection technology mediated by the Gs12-7 protein was evaluated. Using the above targets as the nucleic acid detection sites, the following system reactions were performed: Gs12-7 protein 500 ng, guide RNA 500 ng, 2 μL of 10×CutSmart Buffer, 1 μM of single stranded DNA fluorescence quenching reporter genes (ROX-random-BHQ 2) and 2 μL of PCR amplification target product. The negative control was without a target. The reaction was carried at 37° C., 45° C., 55° C., 60° C., and 65° C. for 15 min separately, and 98° C. for 2 min to inactivate. Fluorescence intensity and background noise were observed under blue light. As shown in FIG. 8, the optimal enzyme digestion reaction temperature for the Gs12-7 protein is 37° C.-60° C., which has relatively high temperature tolerance compared to the known LbCas12a.

Finally, to verify whether the PAM identified by the Gs12-7 protein in bacteria is suitable for nucleic acid detection, target double stranded DNA (dsDNA) was used as the p72 conserved gene of African swine fever virus ASFV, its sequence: CTGTAACGCAGCACAGCTGAACCGTTCTGAAGAAGAAGAAAGTTAATAGCAGATG CCGATACCACAAGATCAGCCGTAGTGATAGACCCCACGTAATCCGTGTCCCAACTA ATATAAAATTCTCTTGCTCTGGATACGTTAATATGACCACTGGGTTGGTATTCCTCCC GTGGCTTCAAAGCAAAGGTAATCATCATCGCACCCGGATCATCGGGGGTTTTAATC GCATTGCCTCCGTAGTGGAAGGGTATGTAAGAGCTGCAGAACTTTGATGGAAATTT ATCGATAAGATTGATACCATGAGCAGTTACGGAAATGTTTTTAATAATAGGTAATGT GATCGGATACGTAACGGGGCTAATATCAGATATAGATGAACATGCGTCTGGAAGAG CTGTATCTCTATCCTGAAAGCTTATCTCTGCGTGGTGAGTGGGCTGCATAATGGCGT TAACAACATGTCCGAACTTGTGCCAATCTCGGTGTTGATGAGGATTTTGATCGGAG ATGTTCCAGGTAGGTTTTAATCCTATAAACATATATTCAATGGGCCATTTAAGAGCA GACATTAGTTTTTCATCGTGGTGGTTATTGTTGGTGTGGGTCACCTGCGTTTTATGG ACACGTATCAGCGAAAAGCGAACGCGTTTTACAAAAAGGTTGTGTATTTCAGGGG TTACAAACAGGTTATTGATGTAAAGTTCATTATTCGTGAGCGAGATTTCATTAATGA CTCCTGGGATAAACCATGG (SEQ ID NO: 61); multiple different guide RNAs (crRNAs) targeting the PAM site of “BTYV” were designed, including crRNA-ATTV-1, crRNA-ATTV-3, crRNA-TTTV-1, crRNA-TTTV-2, crRNA-TTTV-3, crRNA-CTTV-1, crRNA-CTTV-2, crRNA-CTTV-3, crRNA-GTTV-1, crRNA-GTTV-2, crRNA-GTTV-3, and crRNA-PC, with the following sequences:

| | crRNA-ATTV-1: |
|---|---|
| (SEQ ID NO: 62) | |
| | AAUUUCUACUAUUGUAGAUUCUCCCGUGGCUUCAAAGCAA; |
| | crRNA-ATTV-3: |
| (SEQ ID NO: 63) | |
| | AAUUUCUACUAUUGUAGAUUAUACCAUGAGCAGUUACGGA; |
| | crRNA-TTTV-1: |
| (SEQ ID NO: 64) | |
| | AAUUUCUACUAUUGUAGAUUAAGCCACGGGAGGAAUACCA; |
| | crRNA-TTTV-2: |
| (SEQ ID NO: 65) | |
| | AAUUUCUACUAUUGUAGAUUCACUACGGAGGCAAUGCGAU; |
| | crRNA-TTTV-3: |
| (SEQ ID NO: 66) | |
| | AAUUUCUACUAUUGUAGAUUCGUAACUGCUCAUGGUAUCA; |
| | crRNA-CTTV-1: |
| (SEQ ID NO: 67) | |
| | AAUUUCUACUAUUGUAGAUUAAAGCAAAGGUAAUCAUCAU; |
| | crRNA-CTTV-2: |
| (SEQ ID NO: 68) | |
| | AAUUUCUACUAUUGUAGAUUGAUGGAAAUUUAUCGAUAAG; |
| | crRNA-CTTV-3: |
| (SEQ ID NO: 69) | |
| | AAUUUCUACUAUUGUAGAUUCAUACCCUUCCACUACGGAG; |
| | crRNA-GTTV-1: |
| (SEQ ID NO: 70) | |
| | AAUUUCUACUAUUGUAGAUUCGGAAATGUUUUUAAUAAUA; |
| | crRNA-GTTV-2: |
| (SEQ ID NO: 71) | |
| | AAUUUCUACUAUUGUAGAUUAUCUAUAUCUGAUAUUAGCC; |
| | crRNA-GTTV-3: |
| (SEQ ID NO: 72) | |
| | AAUUUCUACUAUUGUAGAUUUUAAUAAUAGGUAAUGUGAU; |
| | crRNA-PC: |
| (SEQ ID NO: 30) | |
| | AAUUUCUACUAUUGUAGAUUAGAGCAGACAUUAGUUUUUC. |

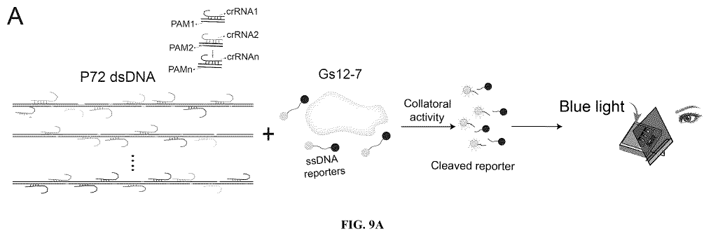

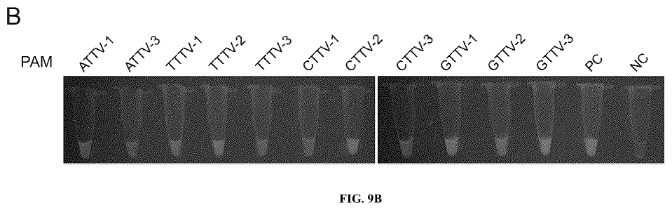

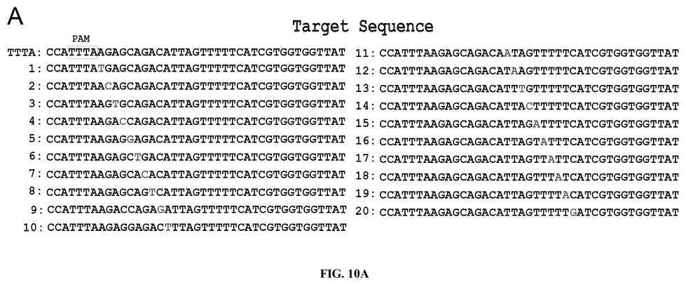

The underline represents the target sequence. Using the above P72 target as the nucleic acid detection site, the above crRNAs were obtained by in vitro transcriptional purification and subjected to the following system reactions: Gs12-7 protein 500 ng, the above different crRNAs 500 ng, 2 μL of 10×CutSmart Buffer, 1 μM of single stranded DNA fluorescence quenching reporter genes (ROX-random-BHQ 2) and 2 μL of PCR amplification product of target P72. The negative control was without a target. The reaction was carried at 37° C. for 15 min, and 98° C. for 2 min to inactivate. The fluorescence intensity of different PAM targets was verified by detecting using a blue light instrument. As shown in FIGS. 9A-9B, all different target sites have high fluorescence signals, indicating that the nucleic acid detection mediated by the Gs12-7 protein can recognize target sites with “BTYV” as PAM.

### Example 5. Evaluating the Specificity of the CRISPR-Gs12-7 System

Further identification of the CRISPR-Gs12-7 system's ability to recognize single base mismatches in the target region. The target double stranded DNA (dsDNA) used in this embodiment is the p72 conserved gene of African swine fever virus ASFV, its sequence:

(SEQ ID NO: 73)
CCATTTAAGAGCAGACATTAGTTTTTCATCGTGGTGGTTATTGTTGGTGT
GGGTCACCTGCGTTTTATGGACACGTATCAGCGAAAAGCGAACGCGTTTT
ACAAAAAGGTTGTGTATTTCAGGGGTTACAAACAGGTTATT,

the bold marking is PAM, and the underline represents the target sequence. Firstly, PCR amplification was performed to obtain a double stranded DNA template containing mutations from the 1-24 consecutive target sites. Target-F to Target-p72-F-20G primers were used upstream, and Target-p72-R primers were used downstream to amplify to obtain the target double stranded gene. The primer sequence table used in this embodiment is as follows:

| Primer name | Sequences (5′-3′) |
|---|---|
| Target-F | CCATTTAAGAGCAGACATTAGTTTTTCA |
| | (SEQ ID NO: 74) |
| Target-PAM-1 | CCAATTAAGAGCAGACATTAGTTTTTCA |
| | (SEQ ID NO: 75) |
| Target-PAM-2 | CCATATAAGAGCAGACATTAGTTTTTCA |
| | (SEQ ID NO: 76) |
| Target-PAM-3 | CCATTAAAGAGCAGACATTAGTTTTTCA |
| | (SEQ ID NO: 77) |
| Target-PAM-4 | CCATTAAAGAGCAGACATTAGTTTTTCA |
| | (SEQ ID NO: 77) |
| Target-p72-F-1T | CCATTTATGAGCAGACATTAGTTTTTCA |
| | (SEQ ID NO: 78) |
| Target-p72-F-2C | CCATTTAACAGCAGACATTAGTTTTTCA |
| | (SEQ ID NO: 79) |
| Target-p72-F-3T | CCATTTAAGTGCAGACATTAGTTTTTCA |
| | (SEQ ID NO: 80) |
| Target-p72-F-4C | CCATTTAAGACCAGACATTAGTTTTTCA |
| | (SEQ ID NO: 81) |
| Target-p72-F-5G | CCATTTAAGAGGAGACATTAGTTTTTCA |
| | (SEQ ID NO: 82) |
| Target-p72-F-6T | CCATTTAAGAGCTGACATTAGTTTTTCA |
| | (SEQ ID NO: 83) |
| Target-p72-F-7C | CCATTTAAGAGCACACATTAGTTTTTCA |
| | (SEQ ID NO: 84) |
| Target-p72-F-8T | CCATTTAAGAGCAGTCATTAGTTTTTCA |
| | (SEQ ID NO: 85) |
| Target-p72-F-9G | CCATTTAAGAGCAGAGATTAGTTTTTCA |
| | (SEQ ID NO: 86) |
| Target-p72-F-10T | CCATTTAAGAGCAGACTTTAGTTTTTCA |
| | (SEQ ID NO: 87) |
| Target-p72-F-11A | CCATTTAAGAGCAGACAATAGTTTTTCA |
| | TCGTGGTG (SEQ ID NO: 88) |
| Target-p72-F-12A | CCATTTAAGAGCAGACATAAGTTTTTCA |
| | TCGTGGTG (SEQ ID NO: 89) |
| Target-p72-F-13T | CCATTTAAGAGCAGACATTTGTTTTTCA |
| | TCGTGGTG (SEQ ID NO: 90) |
| Target-p72-F-14C | CCATTTAAGAGCAGACATTACTTTTTCA |
| | TCGTGGTG (SEQ ID NO: 91) |
| Target-p72-F-15A | CCATTTAAGAGCAGACATTAGATTTTCA |
| | TCGTGGTG (SEQ ID NO: 92) |
| Target-p72-F-16A | CCATTTAAGAGCAGACATTAGTATTTCA |
| | TCGTGGTG (SEQ ID NO: 93) |
| Target-p72-F-17A | CCATTTAAGAGCAGACATTAGTTATTCA |
| | TCGTGGTG (SEQ ID NO: 94) |
| Target-p72-F-18A | CCATTTAAGAGCAGACATTAGTTTATCA |
| | TCGTGGTG (SEQ ID NO: 95) |
| Target-p72-F-19A | CCATTTAAGAGCAGACATTAGTTTTACA |
| | TCGTGGTG (SEQ ID NO: 96) |
| Target-p72-F-20G | CCATTTAAGAGCAGACATTAGTTTTTGA |
| | TCGTGGTG (SEQ ID NO: 97) |
| Target-p72-R | CAATAACCTGTTTGTAACCCCTGAAATA |
| | C (SEQ ID NO: 98) |

Among them, the guide RNA sequence is:

(SEQ ID NO: 30, the underlined area
is the target area)
AAUUUCUACUAUUGUAGAUUAGAGCAGACAUUAGUUUUUC.

The single stranded DNA fluorescence quenching reporter gene sequence is ROX-random-BHQ 2; firstly, the Gs12-7 protein was obtained by prokaryotic expression purification, guide RNA was obtained by in vitro transcription, and the target gene DNA with p72 single base mutation was obtained by PCR amplification. Next, the following reaction system was adopted: Gs12-7 protein 500 ng, guide RNA 500 ng, 2 μL of 10×CutSmart Buffer, 1 μM of single stranded DNA fluorescence quenching reporter genes 5′ROX/GTATCCAGTGCG/3′BHQ2 (SEQ ID NO: 60) and 2 μL of PCR amplification target products with different base mutations. The recognition ability of Gs12-7 protein for single base mismatch sites with the target was evaluated by interpreting fluorescence intensity and background signal under blue light, and its target recognition specificity was evaluated accordingly.

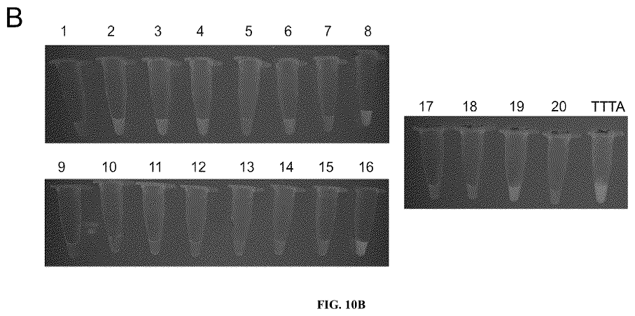

As shown in FIGS. 10A-10B, compared with a completely paired positive target control, the site with a single base mismatch can significantly inhibit the activity of nucleic acid trans-cleavage of the Gs12-7 protein, especially when the single base mutation site is 9-14, its inhibitory effect is significant. From this, it can be seen that the Gs12-7 protein has a strong ability to distinguish single base mismatches in target DNA, indicating its high specificity and suitability as a tool enzyme for single nucleotide sequence polymorphism (SNP) detection or base editing.

### Example 6. CRISPR-Gs12-7 System Mediated Efficient Genome Editing in Eukaryotic Cells

The cell genome directed editing ability mediated by the Gs12-7 protein was evaluated. This embodiment first referred to the instructions of the LipofectamineTMCRISPRMAXTM reagent and incubated the new Gs12-7 and enAsCas12a proteins with guide RNA. Subsequently, ribonucleoprotein complexes (RNPs) were transfected into human HEK 293T cells, and guided by guide RNA, the Gs12-7 and enAsCas12a proteins were recognized and bound to target nucleic acids for genome cleavage. Finally, cells were collected and genomic DNA was extracted, and cleavage activity was detected through T7EN1 digestion.

In this embodiment, the selected target nucleic acid is the human FANCF gene, PAM is TTTG, its sequence:

(SEQ ID NO: 99)
GCCCTACATCTGCTCTCCCTCCACTAAGAAGAACCTCTTTGTGTGGCGAA
AGTAAAAGTATTAGGGCTTTTAAGTTGCCCAGAGTCAAGGAACACGGATA
AAGACGCTGGGAGATTGACATGCATTTCGACCAATAGCATTGCAGAGAGG
CGTATCATTTCGCGGATGTTCCAATCAGTACGCAGAGAGTCGCCGTCTCC
AAGGTGAAAGCGGAAGTAGGGCCTTCGCGCACCTCATGGAATCCCTTCTG
CAGCACCTGGATCGCTTTTCCGAGCTTCTGGCGGTCTCAAGCACTACCTA
CGTCAGCACCTGGGACCCCGCCACCGTGCGCCGGGCCTTGCAGTGGGCGC
GCTACCTGCGCCACATCCATCGGCGCTTTGGTCGGCATGGCCCCATTCGC
ACGGCTCTGGAGCGGCGGCTGCACAACCAGTGGAGGCAAGAGGGCGGCTT
TGGGCGGGGTCCAGTTCCGGGATTAGCGAACTTCCAGGCCCTCGGTCACT
GTGACGTCCTGCTCTCTCTGCGCCTGCTGGAGAACCGGGCCCTCGGGGAT
GCAGCTCGTTACCACCTGGTGCAGCAACT.

the bold part is the PAM sequence, the underlined area is the target area. The guide RNA sequence is:

(SEQ ID NO: 100, the underlined area
is the target area)
AAUUUCUACUAUUGUAGAUUGUCGGCAUGGCCCCAUUCGC;

when the fusion degree of HEK 293T cells reached 70-80%, they were planked and inoculated into a 12 well plate at 8×10⁴ cells/well. Transfection was carried out after 6-8 hours of planking, and 1.25 μg of the predicted Genie scissor or Cas12a-NLS-tagged protein was added and incubated with 625 ng of guide RNA, mixed well with 50 μL of opti-MEM and 2.6 μL of Cas9 Plus™ reagent; 50 μL of opti-MEM was mixed well with 3 μL of CRISPR™ reagent. Diluted CRISPR™ reagent was mixed well with diluted RNP and incubated at room temperature for 10 min. The incubated mixture was added to the culture medium covered with cells for transfection. After incubating at 37° C. for 72 hours, the culture medium was discarded and 100 μL of PBS was used to perform cell resuspension to extract the genome of cells. PCR amplification was performed on the target sites of transfected positive cells. T7EN1 enzyme treatment reaction and agarose gel electrophoresis were used to observe the changes of bands to judge whether the predicted protein had gene editing activity in vivo, and Image J was used to roughly calculate the editing efficiency. The template for negative control is the genome of normal cultured HEK 293T cells without RNP transfection.

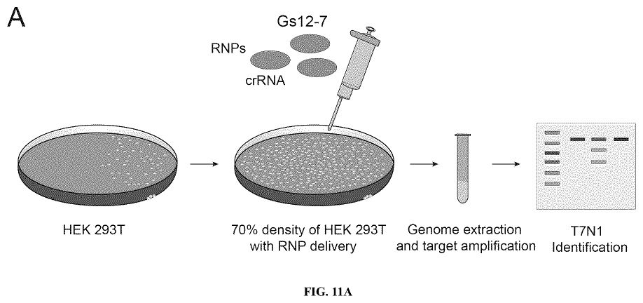

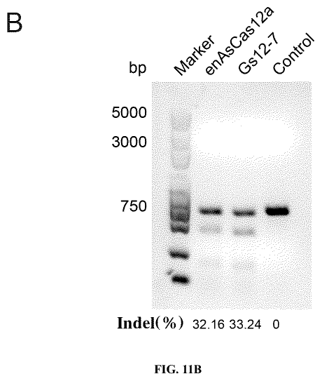

As shown in FIGS. 11A-11B, compared with the negative control without RNP transfection, the enAsCas12a and Gs12-7 proteins in the experimental group show significant cell genome editing activity through T7EN1 digestion reaction and electrophoresis detection. Their cleaving efficiency (Index) are 32.16% and 33.14%, respectively. It can be seen that the newly discovered Gs12-7 protein can be used for cell genome directed or specific editing, and the editing activity is consistent with the enhanced enAsCas12a activity.

Further, in this embodiment, the eukaryotic cell codon of the newly discovered Gs12-7 protein was optimized, and SV40 NLS and NLS nuclear localization signals were added to the N and C terminals of its protein, respectively. The sequence was shown in SEQ ID NO: 4. The synthesized sequence was constructed into Lenti-puro lentivirus vector, and at the same time, it was co-transfected with the guide RNA eukaryotic expression vector to HEK293T cells through liposomes. The guide RNA paired with the target nucleic acid was used to guide the Gs12-7 protein to recognize and cleave the target nucleic acid molecule, and whether it had cell genome directed editing activity was detected through T7EN1 digestion and agarose gel electrophoresis.

In this embodiment, the selected target nucleic acid is human FANCF gene, PAM is TTTG, its sequence:

(SEQ ID NO: 101)
GCCCTACATCTGCTCTCCCTCCACTAAGAAGAACCTCTTTGTGTGGCGAA
AGTAAAAGTATTAGGGCTTTTAAGTTGCCCAGAGTCAAGGAACACGGATA
AAGACGCTGGGAGATTGACATGCATTTCGACCAATAGCATTGCAGAGAGG
CGTATCATTTCGCGGATGTTCCAATCAGTACGCAGAGAGTCGCCGTCTCC
AAGGTGAAAGCGGAAGTAGGGCCTTCGCGCACCTCATGGAATCCCTTCTG
CAGCACCTGGATCGCTTTTCCGAGCTTCTGGCGGTCTCAAGCACTACCTA
CGTCAGCACCTGGGACCCCGCCACCGTGCGCCGGGCCTTGCAGTGGGCGC
GCTACCTGCGCCACATCCATCGGCGCTTTGGTCGGCATGGCCCCATTCGC
ACGGCTCTGGAGCGGCGGCTGCACAACCAGTGGAGGCAAGAGGGCGGCTT
TGGGCGGGGTCCAGTTCCGGGATTAGCGAACTTCCAGGCCCTCGGTCACT
GTGACGTCCTGCTCTCTCTGCGCCTGCTGGAGAACCGGGCCCTCGGGGAT
GCAGCTCGTTACCACCTGGTGCAGCAACTCTTTCCCGGCCC;

the bold part is the PAM sequence, the underlined area is the target area, and the guide RNA sequence is:

(SEQ ID NO: 100, the underlined area
is the target area)
AAUUUCUACUAUUGUAGAUUGUCGGCAUGGCCCCAUUCGC;

and the human RUNX1 gene, PAM is TTTC, its sequence:

(SEQ ID NO: 102)
CATCACCAACCCACAGCCAAGGCGGCGCTGGCTTTTTTTTTTTTTTTAAT
CTTTAACAATTTGAATATTTGTTTTTACAAAGGTGCATTTTTTAATAGGG
CTTGGGGAGTCCCAGAGGTATCCAGCAGAGGGGAGAAGAAAGAGAGATGT
AGGGCTAGAGGGGTGAGGCTGAAACAGTGACCTGTCTTGGTTTTCGCTCC
GAAGGTAAAAGAAATCATTGAGTCCCCCGCCTTCAGAAGAGGGTGCATTT
TCAGGAGGAAGCGATGGCTTCAGACAGCATATTTGAGTCATTTCCTTCGT
ACCCACAGTGCTTCATGAGAGGTGAGTACATGCTGGTCTTGTAATATCTA
CTTTTGCTCAGCTTTGCCTGTAATGAAATGGCAGCTTGTTTCACCTCGGT
GCAGAGATGCCTCGGTGCCTGCCAGTTCCCTGTCTTGTTTGTGAGAGGAA
TTCAAACTGAGGCATATGATTACAAGTCTATTGGATTACTTACTAATCAG
ATGGAAGCTCTTCAGAAATGTTTTAATAAATACTTAGTTATGCTGTTGGA
GTGTTC,

the bold part is the PAM sequence, the underlined area is the target area; the human EMX1 gene, PAM is TTTG, its sequence:

(SEQ ID NO: 103)
GGAGCAGCTGGTCAGAGGGGACCCCGGCCTGGGGCCCCTAACCCTATGTA
GCCTCAGTCTTCCCATCAGGCTCTCAGCTCAGCCTGAGTGTTGAGGCCCC
AGTGGCTGCTCTGGGGGCCTCCTGAGTTTCTCATCTGTGCCCCTCCCTCC
CTGGCCCAGGTGAAGGTGTGGTTCCAGAACCGGAGGACAAAGTACAAACG
GCAGAAGCTGGAGGAGGAAGGGCCTGAGTCCGAGCAGAAGAAGAAGGGCT
CCCATCACATCAACCGGTGGCGCATTGCCACGAAGCAGGCCAATGGGGAG
GACATCGATGTCACCTCCAATGACTAGGGTGGGCAACCACAAACCCACGA
GGGCAGAGTGCTGCTTGCTGCTGGCCAGGCCCCTGCGTGGGCCCAAGCTG
GACTCTGGCCACTCCCTGGCCAGGCTTTGGGGAGGCCTGGAGTCATGGCC
CCACAGGGCTTGAAGCCCGGGGCCGCCATTGACAGAGGGACAAGCAATGG
GCTGGCTGAGGCCTGGGACCACTTGGCCTTCTCCTCGGAGAGCCTGCCTG
CCTGGGCGGGCCCGCCCGCCACCGCAGCCTCCCAGCTGCTCTCCGTGTCT
CCAATCTCCCTTTTGTTTTGATGCATTTCTGTTTTAATTTATTTTCCAGG
CACCACTGTAGTTTAGTGATCCCCAGTGTCCCCCTTCCCTATGG,

the two designed guide RNA sequences are:

| | E-crRNA1, |
|---|---|
| (SEQ ID NO: 104) | |
| | AAUUUCUACUAUUGUAGAUUUGGUUGCCCACCCUAGUCAU; |
| | E-crRNA2, |
| | (SEQ ID NO: 105, the underlined |
| | area is the target area) |
| | AAUUUCUACUAUUGUAGAUUUACUUUGUCCUCCGGUUCUG. |

When the fusion degree of HEK 293T cells reached 70%-80%, they were planked and inoculated into a 12 well plate at 8×10⁴ cells/well. Transfection was carried out after 6-8 hours of planking, and 1 μg of eukaryotic expression vector or known enhanced enAsCas12a eukaryotic expression vector, 1 μg of single or tandem guide RNA expression vectors and 10 μL of Jetprime regent were added in turn to 200 μL of Jetprime Buffer to pipette and incubate at room temperature for 10 min. The incubated mixture was added to the culture medium covered with cells for transfection. After incubating at 37° C. for 72 hours, the culture medium was discarded and 100 μL of PBS was used to perform cell resuspension to extract the genome of cells. PCR amplification was performed on the target site of transfected positive cells to edit the nearby sequences. The changes of target bands were observed by T7EN1 digestion and agarose gel electrophoresis. The negative control template was the normal culture HEK293 cell genome without transfection.

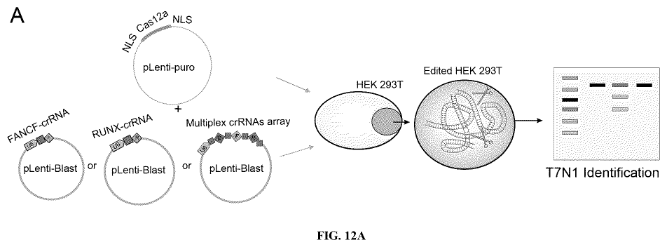

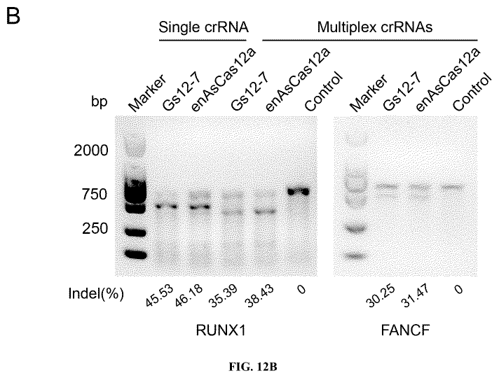

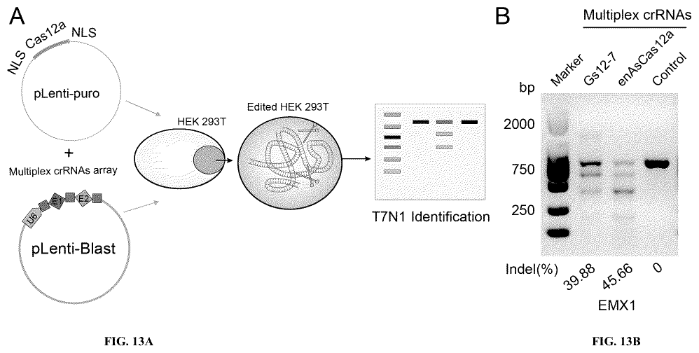

The directed editing ability of CRISPR-Gs12-7 protein towards a single target gene, multiple target genes, and multiple single gene loci was evaluated. As shown in FIGS. 12A-13B, when editing a single site of the RUNX1 gene, it is found that the cleavage activity of newly identified Gs12-7 and known enAsCas12a are 45.53% and 46.18%, respectively, with similar activity (FIGS. 12A-12B). When editing both RUNX1 and FANCF simultaneously, it is found that the editing efficiency of Gs12-7 and known enAsCas12a for the RUNX1 gene is 35.39% and 38.43%, respectively, while for the FANCF gene, their editing activities are 30.25% and 31.45%, respectively. In FIGS. 13A-13B, when editing two loci of the EMX1 gene simultaneously, the editing activities of Gs12-7 and the known enAsCas12a are 39.88% and 45.66%, respectively. It can be seen that the newly identified Gs12-7 protein can achieve single or multiple gene editing, and its activity is consistent with the enhanced enAsCas12a.

The above are only preferred embodiments of the present invention, and are not intended to limit the present invention in any form. Although the present invention has been disclosed in the above preferred embodiments, they are not intended to limit the present invention. Any technician who is familiar with the present invention can make a slight change or modification into the equivalent embodiments of equivalent changes by using the technical content of the above-mentioned hints without departing from the scope of the technical solution of the present invention. But as long as the technical content is not deviated from the technical solution of the present invention, any simple modifications, equivalent changes and modifications made to the above embodiments according to the technical substance of the present invention are still within the scope of the present invention.

What is claimed is:

1. An endonuclease in a clustered regularly interspaced short palindromic repeats (CRISPR)/CRISPR associated (Cas) system, wherein the endonuclease is a Gs12-7 protein with the amino acid sequence as shown in SEQ ID NO: 1.

2. A polynucleotide, wherein the polynucleotide encodes the endonuclease of claim 1.

3. A vector, wherein the vector comprises the polynucleotide of claim 2.

4. A host cell, wherein the host cell comprises the polynucleotide of claim 2 or a vector comprising the polynucleotide, and the host cell is not a plant cell.

5. A method of gene editing, comprising using the endonuclease of claim 1, or a polynucleotide encoding the endonuclease, or a vector comprising the polynucleotide, or a host cell comprising the polynucleotide or the vector, wherein the host cell is not a plant cell.

6. The method of claim 5, wherein the gene editing includes gene modification or gene knockout of prokaryotic and eukaryotic genomes.

7. A CRISPR/Cas gene editing system, comprising the endonuclease of claim 1, or a polynucleotide encoding the endonuclease, or a vector comprising the polynucleotide, or a host cell comprising the polynucleotide or the vector, wherein the host cell is not a plant cell.

8. The CRISPR/Cas gene editing system of claim 7, further comprising a direct repeat sequence capable of binding to the endonuclease of claim 1 and a guiding sequence capable of targeting a target sequence.

9. A visual nucleic acid detection kit, comprising the endonuclease of claim 1, a single stranded DNA fluorescence quenching reporter gene, and a guide RNA paired with a target nucleic acid.
